(19) United States
(12) Patent Application Publication
(10) Pub. No.: US 2023/0059825 A1
(43) Pub. Date: Feb. 23, 2023

(54) BACILLUS STRAINS WITH THE ABILITY TO DEGRADE INORGANIC NITROGEN COMPOUNDS

(71) Applicant: EVONIK OPERATIONS GMBH, Essen (DE)

(72) Inventors: Christos GIATSIS, Münster (DE); Michelle DARGATZ, Bielefeld (DE); Lorena STANNEK-GÖBEL, Bielefeld (DE); Stefan PELZER, Gütersloh (DE)

(21) Appl. No.: 17/792,582

(22) PCT Filed: Jan. 6, 2021
(86) PCT No.: PCT/EP2021/050111

§ 371 Date: Jul. 13, 2022

(30) Foreign Application Priority Data
Jan. 14, 2020 (EP) 20151668.9

(51) Int. Cl. C12N 1/20; C02F 3/34

(52) U.S. Cl. CPC C12N 1/205; C02F 3/34; C02F 2101/16

(57) ABSTRACT

The invention concerns new Bacillus strains which are able to degrade effectively inorganic nitrogen compounds and are further able to inhibit the growth of pathogens of aquatic animals.

The invention concerns new Bacillus strains which are able to degrade effectively inorganic nitrogen compounds and are further able to inhibit the growth of pathogens of aquatic animals.

The biological removal of inorganic nitrogen compounds, such as ammonium and nitrate from aquatic systems is a topic of particular interest, as inorganic nitrogen compounds contribute to eutrophication and are toxic to many aquatic organisms. Thus, increased levels of those substances in aquatic systems, in particular in rearing water, are undesirable. In the past, combinations of autotrophic nitrifying and denitrifying bacteria (which convert ammonium to nitrogen, with nitrate as an intermediate) were described for effecting remediation. Meanwhile, also alternative methods have been described like for example in US 2016/0326034, where mixtures of Bacilli and lactic acid bacteria are described for decreasing nitrate levels. In the methods as disclosed in the state of the art, normally consortia of different microorganisms are employed for removal of undesired nitrogen compounds, wherein the different microorganisms fulfil different functions and only the consortia as a whole are able to reach a satisfying result.

According to the invention, it was surprisingly found out that specific bacteria of the genus Bacillus are able to degrade effectively different inorganic nitrogen compounds, in particular selected from ammonium, nitrite and nitrate, even without the aid of further microorganisms. “Degrade” according to the invention means preferably the transformation of those compounds into molecular nitrogen.

It surprisingly turned out further that the identified bacteria are not only able to degrade inorganic nitrogen compounds, but they possess in addition other beneficial characteristics, in particular inhibitory activity against pathogens of aquatic animals, which qualify them as probiotics (also called “direct-fed microbials” or “DFM”).

Thus, basing on the characteristics of the strains, subjects of the present invention are in particular the use of the strains of the invention for degrading inorganic nitrogen compounds, in particular in aquaculture systems, as well as their use as probiotics, in particular in the feed industry, and very preferred in aquaculture. The different subjects of the inventions are explained more in detail further below.

The Bacillus strains DSM 33349, DSM 33350, DSM 33351 and DSM 33352 have been identified by targeted screening of naturally occurring isolates and have been classified as Bacillus subtilis strains. They have been deposited with the DSMZ (Leibniz Institute DSMZ-German Collection of Microorganisms and Cell Cultures, Inhoffenstraße 7B, 38124 Braunschweig, Germany) on Dec. 3, 2019 under the provisions of the Budapest Treaty on the International Recognition of the Deposit of Microorganisms for the Purpose of Patent Procedure under the Accession Numbers as mentioned before in the name of the applicant Evonik Operations GmbH.

Thus, a first subject of the invention is a Bacillus strain, in particular a Bacillus subtilis strain, or a preparation thereof, wherein the strain or preparation are able to degrade at least one inorganic nitrogen compound and are further able to inhibit the growth of at least one pathogen.

According to the invention, the inorganic nitrogen compound, which can be degraded by the strains and preparations of the invention, is preferably selected from ammonium, nitrite and nitrate.

The Bacillus strain, in particular Bacillus subtilis strain, or preparation thereof is according to the invention preferably selected from the following group:
a) a Bacillus strain as deposited under one of the following numbers at the DSMZ: DSM 33349, DSM 33350, DSM 33351, DSM 33352;
b) a mutant of a Bacillus strain as listed in (a) with a sequence identity to the strain as listed in (a) of at least 95%, preferably at least 98%, 99 or 99.5%;
c) a preparation of a strain according to (a) or (b);
d) a preparation comprising an effective mixture of compounds as contained in a strain as listed in (a) or (b) or as contained in a preparation of (c).

The Bacillus strain of the invention has preferably a 16S rDNA sequence with a sequence identity of at least 99%, preferably at least 99.5 or 99.8%, above all 100%, to a sequence according to SEQ ID NO: 1 (in particular strain DSM 33349 or a mutant thereof), SEQ ID NO: 6 (in particular strain DSM 33350 or a mutant thereof), SEQ ID NO: 11 (in particular strain DSM 33351 or a mutant thereof) or SEQ ID NO: 16 (in particular strain DSM 33352 or a mutant thereof).

In a preferred embodiment of the invention, the Bacillus strain, in particular the strain DSM 33349 or a mutant thereof, exhibits at least one, in particular at least 2, 3 or 4, preferably all, of the following characteristics:
a) a 16S rDNA sequence with a sequence identity of at least 99.5%, preferably at least 99.8%, above all 100%, to the polynucleotide sequence according to SEQ ID NO: 1 and/or to the 16S rDNA sequence of the strain DSM 33349 as deposited with the DSMZ;
b) a yqfD sequence with a sequence identity of at least 99.5%, preferably at least 99.8%, above all 100%, to the polynucleotide sequence according to SEQ ID NO: 2 and/or to the yqfD sequence of the strain DSM 33349 as deposited with the DSMZ;
c) a gyrB sequence with a sequence identity of at least 99.5%, preferably at least 99.8%, above all 100%, to the polynucleotide sequence according to SEQ ID NO: 3 and/or to the gyrB sequence of the strain DSM 33349 as deposited with the DSMZ;
d) an rpoB sequence with a sequence identity of at least 99.5%, preferably at least 99.8%, above all 100%, to the polynucleotide sequence according to SEQ ID NO: 4 and/or to the rpoB sequence of the strain DSM 33349 as deposited with the DSMZ;
e) a groEL sequence with a sequence identity of at least 99.5%, preferably at least 99.8%, above all 100%, to the polynucleotide sequence according to SEQ ID NO: 5 and/or to the groEL sequence of the strain DSM 33349 as deposited with the DSMZ;
f) a recT sequence with a sequence identity of at least 95%, preferably at least 98, 99 or 99.5%, above all 100%, to the polynucleotide sequence according to SEQ ID NO: 21.

In a further preferred embodiment of the invention, the Bacillus strain, in particular the strain DSM 33350 or a mutant thereof, exhibits at least one, in particular at least 2, 3 or 4, preferably all, of the following characteristics:
a) a 16S rDNA sequence with a sequence identity of at least 99.5%, preferably at least 99.8%, above all 100%, to the polynucleotide sequence according to SEQ ID NO: 6 and/or to the 16S rDNA sequence of the strain DSM 33350 as deposited with the DSMZ;
b) a yqfD sequence with a sequence identity of at least 99.5%, preferably at least 99.8%, above all 100%, to the polynucleotide sequence according to SEQ ID NO: 7 and/or to the yqfD sequence of the strain DSM 33350 as deposited with the DSMZ;
c) a gyrB sequence with a sequence identity of at least 99.5%, preferably at least 99.8%, above all 100%, to the polynucleotide sequence according to SEQ ID NO: 8 and/or to the gyrB sequence of the strain DSM 33350 as deposited with the DSMZ;
d) an rpoB sequence with a sequence identity of at least 99.5%, preferably at least 99.8%, above all 100%, to the polynucleotide sequence according to SEQ ID NO: 9 and/or to the rpoB sequence of the strain DSM 33350 as deposited with the DSMZ;
e) a groEL sequence with a sequence identity of at least 99.5%, preferably at least 99.8%, above all 100%, to the polynucleotide sequence according to SEQ ID NO: 10 and/or to the groEL sequence of the strain DSM 33350 as deposited with the DSMZ.

In a further preferred embodiment of the invention, the Bacillus strain, in particular the strain DSM 33351 or a mutant thereof, exhibits at least one, in particular at least 2, 3 or 4, preferably all, of the following characteristics:
a) a 16S rDNA sequence with a sequence identity of at least 99.5%, preferably at least 99.8%, above all 100%, to the polynucleotide sequence according to SEQ ID NO: 11 and/or to the 16S rDNA sequence of the strain DSM 33351 as deposited with the DSMZ;
b) a yqfD sequence with a sequence identity of at least 99.5%, preferably at least 99.8%, above all 100%, to the polynucleotide sequence according to SEQ ID NO: 12 and/or to the yqfD sequence of the strain DSM 33351 as deposited with the DSMZ;
c) a gyrB sequence with a sequence identity of at least 99.5%, preferably at least 99.8%, above all 100%, to the polynucleotide sequence according to SEQ ID NO: 13 and/or to the gyrB sequence of the strain DSM 33351 as deposited with the DSMZ;
d) an rpoB sequence with a sequence identity of at least 99.5%, preferably at least 99.8%, above all 100%, to the polynucleotide sequence according to SEQ ID NO: 14 and/or to the rpoB sequence of the strain DSM 33351 as deposited with the DSMZ;
e) a groEL sequence with a sequence identity of at least 99.5%, preferably at least 99.8%, above all 100%, to the polynucleotide sequence according to SEQ ID NO: 15 and/or to the groEL sequence of the strain DSM 33351 as deposited with the DSMZ;
f) a sequence with a sequence identity of at least 95%, preferably at least 98, 99 or 99.5%, above all 100%, to the polynucleotide sequence according to SEQ ID NO: 22 (encoding a hypothetical gamma-polyglutamate hydrolase PghZ);
g) a sequence with a sequence identity of at least 95%, preferably at least 98, 99 or 99.5%, above all 100%, to the polynucleotide sequence according to SEQ ID NO: 23 (encoding a hypothetical protein).

In a further preferred embodiment of the invention, the Bacillus strain, in particular the strain DSM 33352 or a mutant thereof, exhibits at least one, in particular at least 2, 3 or 4, preferably all, of the following characteristics:
a) a 16S rDNA sequence with a sequence identity of at least 99.5%, preferably at least 99.8%, above all 100%, to the polynucleotide sequence according to SEQ ID NO: 16 and/or to the 16S rDNA sequence of the strain DSM 33352 as deposited with the DSMZ;
b) a yqfD sequence with a sequence identity of at least 99.5%, preferably at least 99.8%, above all 100%, to the polynucleotide sequence according to SEQ ID NO: 17 and/or to the yqfD sequence of the strain DSM 33352 as deposited with the DSMZ;
c) a gyrB sequence with a sequence identity of at least 99.5%, preferably at least 99.8%, above all 100%, to the polynucleotide sequence according to SEQ ID NO: 18 and/or to the gyrB sequence of the strain DSM 33352 as deposited with the DSMZ;
d) an rpoB sequence with a sequence identity of at least 99.5%, preferably at least 99.8%, above all 100%, to the polynucleotide sequence according to SEQ ID NO: 19 and/or to the rpoB sequence of the strain DSM 33352 as deposited with the DSMZ;
e) a groEL sequence with a sequence identity of at least 99.5%, preferably at least 99.8%, above all 100%, to the polynucleotide sequence according to SEQ ID NO: 20 and/or to the groEL sequence of the strain DSM 33352 as deposited with the DSMZ;
f) a sequence with a sequence identity of at least 95%, preferably at least 98, 99 or 99.5%, above all 100%, to the polynucleotide sequence according to SEQ ID NO: 24 (encoding a hypothetical phage tail tape measure protein);
g) a sequence with a sequence identity of at least 95%, preferably at least 98, 99 or 99.5%, above all 100%, to the polynucleotide sequence according to SEQ ID NO: 25 (encoding a hypothetical protein).

Thus, a particular subject of the invention is also a Bacillus strain, preferably a B. subtilis strain, in particular the strain DSM 33349 or a mutant thereof, exhibiting the following characteristic:

a) a 16S rDNA sequence according to SEQ ID NO: 1 and/or a 16S rDNA sequence of the strain DSM 33349 as deposited with the DSMZ;

Preferably, this Bacillus strain exhibits at least one, two or three, preferably all, further characteristic(s) as follows:

b) a yqfD sequence according to SEQ ID NO: 2 and/or a yqfD sequence of the strain DSM 33349 as deposited with the DSMZ;
c) a gyrB sequence according to SEQ ID NO: 3 and/or a gyrB sequence of the strain DSM 33349 as deposited with the DSMZ;
d) an rpoB sequence according to SEQ ID NO: 4 and/or a rpoB sequence of the strain DSM 33349 as deposited with the DSMZ;
e) a groEL sequence according to SEQ ID NO: 5 and/or a groEL sequence of the strain DSM 33349 as deposited with the DSMZ;
f) a recT sequence according to SEQ ID NO: 21.

Thus, a particular subject of the invention is also a Bacillus strain, preferably a B. subtilis strain, in particular the strain DSM 33350 or a mutant thereof, exhibiting the following characteristic:

a) a 16S rDNA sequence according to SEQ ID NO: 6 and/or a 16S rDNA sequence of the strain DSM 33350 as deposited with the DSMZ;

Preferably, this Bacillus strain exhibits at least one, two or three, preferably all, further characteristic(s) as follows:

b) a yqfD sequence according to SEQ ID NO: 7 and/or a yqfD sequence of the strain DSM 33350 as deposited with the DSMZ;
c) a gyrB sequence according to SEQ ID NO: 8 and/or a gyrB sequence of the strain DSM 33350 as deposited with the DSMZ;
d) an rpoB sequence according to SEQ ID NO: 9 and/or an rpoB sequence of the strain DSM 33350 as deposited with the DSMZ;
e) a groEL sequence according to SEQ ID NO: 10 and/or a groEL sequence of the strain DSM 33350 as deposited with the DSMZ.

Thus, a particular subject of the invention is also a Bacillus strain, preferably a B. subtilis strain, in particular the strain DSM 33351 or a mutant thereof, exhibiting the following characteristic:

a) a 16S rDNA sequence according to SEQ ID NO: 11 and/or a 16S rDNA sequence of the strain DSM 33351 as deposited with the DSMZ;

Preferably, this Bacillus strain exhibits at least one, two or three, preferably all, further characteristic(s) as follows:

b) a yqfD sequence according to SEQ ID NO: 12 and/or a yqfD sequence of the strain DSM 33351 as deposited with the DSMZ;
c) a gyrB sequence according to SEQ ID NO: 13 and/or a gyrB sequence of the strain DSM 33351 as deposited with the DSMZ;
d) an rpoB sequence according to SEQ ID NO: 14 and/or a rpoB sequence of the strain DSM 33351 as deposited with the DSMZ;
e) a groEL sequence according to SEQ ID NO: 15 and/or a groEL sequence of the strain DSM 33351 as deposited with the DSMZ;
f) a sequence according to SEQ ID NO: 22;
e) a sequence according to SEQ ID NO: 23.

Thus, a particular subject of the invention is also a Bacillus strain, preferably a B. subtilis strain, in particular the strain DSM 33352 or a mutant thereof, exhibiting the following characteristic:

a) a 16S rDNA sequence according to SEQ ID NO: 16 and/or a 16S rDNA sequence of the strain DSM 33352 as deposited with the DSMZ;

Preferably, this Bacillus strain exhibits at least one, two or three, preferably all, further characteristic(s) as follows:

b) a yqfD sequence according to SEQ ID NO: 17 and/or a yqfD sequence of the strain DSM 33352 as deposited with the DSMZ;
c) a gyrB sequence according to SEQ ID NO: 18 and/or a gyrB sequence of the strain DSM 33352 as deposited with the DSMZ;
d) an rpoB sequence according to SEQ ID NO: 19 and/or a rpoB sequence of the strain DSM 33352 as deposited with the DSMZ;
e) a groEL sequence according to SEQ ID NO: 20 and/or a groEL sequence of the strain DSM 33352 as deposited with the DSMZ;
f) a sequence according to SEQ ID NO: 24;
g) a sequence according to SEQ ID NO: 25.

The terms “mutant” or “variant”, if not explicitly described otherwise, according to the invention relate to strains with a sequence identity of at least 95%, 96% or 97%, preferably at least 98%, 99 or 99.5% more preferably at least 99.8 or 99.9%, with respect to the genomic DNA of the parent strain. I.e. in this context the term “sequence identity” always relates to the complete genomic DNA, if not explicitly mentioned otherwise.

A further subject of the invention is a composition containing at least two Bacillus strains or preparations thereof according to the invention, wherein the Bacillus strains and preparations thereof are preferably selected from the following group:
a) a Bacillus strain as deposited under one of the following numbers at the DSMZ: DSM 33349, DSM 33350, DSM 33351 and DSM 33352;
b) a mutant of a Bacillus strain as listed in (a) with a sequence identity to the strain as listed in (a) of at least 95%, preferably at least 98%, 99 or 99.5%;
c) a preparation of a strain according to (a) or (b);
d) a preparation comprising an effective mixture of compounds as contained in a strain as listed in (a) or (b) or as contained in a preparation of (c).

Very preferred according to the invention are compositions which contain a combination of the strains DSM 33351 and DSM 33352.

The bacteria of the invention are able to degrade inorganic nitrogen compounds, preferably at least one, more preferably at least two, inorganic nitrogen compounds selected from ammonium, nitrite and nitrate.

Thus, a further subject of the invention is a method of decreasing the amount of inorganic nitrogen compounds and/or controlling the amount of inorganic nitrogen compounds in an aqueous system, wherein the inorganic nitrogen compounds are preferably selected from ammonium, nitrite and nitrate, very preferably from ammonium and nitrite, the method comprising supplying the aqueous system with at least one Bacillus strain or preparation thereof according to the invention, in particular with at least Bacillus subtilis strain or preparation thereof, or with a composition according to the invention, wherein the Bacillus strain and/or preparation thereof and/or composition are able to degrade at least one inorganic nitrogen compound and are further able to inhibit the growth of at least one pathogen, in particular of at least one pathogen of aquatic animals, wherein the Bacillus strains and preparations, in particular Bacillus strains and preparations of the composition, are preferably selected from the following group:
a) a Bacillus strain, in particular B. subtilis strain, as deposited under one of the following numbers at the DSMZ: DSM 33349, DSM 33350, DSM 33351, DSM 33352;
b) a mutant of a Bacillus strain as listed in (a) with a sequence identity to the strain as listed in (a) of at least 95%, preferably at least 98%, 99 or 99.5%;
c) a preparation of a strain according to (a) or (b);
d) a preparation comprising an effective mixture of compounds as contained in a strain as listed in (a) or (b) or as contained in a preparation of (c).

The aqueous system according to the invention can be any kind of aqueous system, in particular a natural or artificial aqueous system like a lake, a pond, a basin, an ornamental pond, aquaria, aquaculture facilities or segregated areas in the sea, in lakes or in rivers. Preferably the aqueous system is a water reservoir which is used for rearing animals, in particular for rearing aquatic animals. Thus, the aqueous system is very preferably drinking water or rearing water, in particular for rearing aquatic animals. Depending on the kind of animal to be raised, the water can be saltwater as well as freshwater.

As bacteria which are able to degrade inorganic nitrogen compounds and at the same time are suitable as probiotics have not been disclosed before in the state of the art, thus, a further subject of the invention are bacteria, in particular of the genus Bacillus, preferably of the species Bacillus subtilis, which are able to degrade inorganic nitrogen compounds and at the same time exhibit probiotic activity, in particular with respect to aquatic animals, wherein the bacteria preferably comprise further characterizing features as disclosed in this application, in particular characterizing DNA sequences as mentioned above.

Being able to degrade inorganic nitrogen compounds according to the invention preferably means that the bacteria are able to remove at least 50%, more preferably at least 80 or 90%, above all more than 95%, of ammonia and/or nitrate under at least one test condition, preferably under all test conditions, as depicted in working example 1.

The bacteria according to the invention are preferably able to effectively degrade inorganic nitrogen compounds also in environments where the C:N ratio is very low, in particular in C minimal media. In particular they are preferably able to effectively degrade inorganic nitrogen compounds in environments, where the C:N ratio is below 100, in particular between 20 and 100, between 30 and 90 or between 40 and 80.

The bacteria according to the invention are preferably able to grow under high salt conditions, in particular in presence of 1 wt.-% of NaCl, more preferably in presence of 1.5 or 2 wt.-% of NaCl, above all in presence of 2.5 or 3 wt.-% of NaCl, for at least one day. Further the bacteria according to the invention are preferably able to effectively degrade inorganic nitrogen compounds, in particular selected from ammonium, nitrite and nitrate, in aqueous environments with such high amounts of sodium chloride.

The spores of the bacteria of the present invention are preferably able to germinate effectively in C minimal media, i.e. in environments, where the amount of carbon sources is relatively low. Preferably the C minimal medium is a medium where the amount of carbon sources is below 50 g per kg, more preferably below 25 g per kg, in particular in the range of 10 to 50 g or 15 to 25 g carbon source per kg medium. Preferably the spores of the invention outgrow in such minimal media within not more than 30 hours, more preferably within not more than 25 hours.

The bacteria of the invention are further preferably able to inhibit at least one pathogenic microorganism, in particular at least one pathogenic bacterium, wherein the at least one pathogenic microorganism or bacterium is preferably pathogenic for an aquatic animal.

Very preferably the bacteria of the invention are able to inhibit at least one bacterium selected from the group consisting of Vibrio harveyi, Vibrio parahaemolyticus, Aeromonas hydrophila and/or Streptococcus agalactiae, with inhibition of Streptococcus agalactiae being preferred.

Being able to inhibit a pathogen according to the invention preferably means that the strain is able to cause a pathogen clearance of at least 3 mm, preferably at least 5 or 10 mm, with respect to said pathogen in a well diffusion antagonism test according to Parente et al. (1995).

It is particularly preferred according to the invention that the bacteria according to the invention are able to inhibit the growth of Streptococcus agalactiae, in particular the growth of Streptococcus agalactiae DSM 2134, very effectively. In particular they are preferably characterized by a pathogen clearance of at least 10 mm, more preferably at least 15 mm, in a well diffusion antagonism assay on LBKelly agar plates with respect to Streptococcus agalactiae DSM 2134.

The bacteria of the invention are preferably characterized by at least one, more preferably at least two, three or four, of the following further features:

They are preferably able to grow under anaerobic conditions. Further, they are preferably able to degrade water-insoluble cellulose and/or protein under such anaerobic conditions.

Further, the bacteria of the invention are preferably further able to degrade water-insoluble cellulose and/or protein under aerobic conditions, in particular in the presence of 2 mM bile.

The bacteria according to the invention are preferably further characterized by exhibiting at least one, two or three, preferably all, of the following enzymatic activities: cellulase activity; xylanase activity; protease activity; catalase activity; superoxide dismutase activity.

The combined presence of proteolytic activity and the ability to degrade inorganic nitrogen compounds is a very preferred embodiment of the invention, as such combined activities allow a complete removal of undigested feed protein from rearing water and thus represent a desired synergistic effect.

The bacteria of the invention preferably furthermore produce lactate and they are preferably further able to degrade mycotoxins.

The bacteria according to the invention are preferably further characterized by being able to grow in presence of 2 mM bile, more preferably in presence of 4 mM bile, and/or in presence of 0.3 wt.-% porcine bile and/or in presence of 0.3 wt.-% chicken bile. In particular they are preferably characterized by being able to proliferate fast under such high bile concentrations.

Further the bacteria of the invention, i.e. a significant part, in particular at least 80%, of a respective sample, preferably survive the high temperatures necessary for pelleting animal feed, in particular they preferably survive a temperature of 80° C., more preferably of 95 or 99° C., for at least 20 minutes.

Without wishing to be bound by any theory, it is thought that the Bacillus strains according to the invention are able to enhance animal health, in particular gut health and/or the health of aquatic animals, by a multifaceted mode of action, including the production of antibacterial metabolites with selective efficacy and the competition with pathogenic bacteria by better consuming the available nutrients, thereby suppressing effective establishment of pathogenic bacteria, in particular in the gut, in the gills or on the skin. Hereby the enzymes produced by Bacillus may help to establish a balanced microbiota by providing predigested nutrients, in particular in aquatic animals.

It is a big advantage of probiotics in comparison to antibiotics, that they do not attack bacteria indiscriminately nor do they lead to antibiotic resistant strains of pathogenic bacteria. Normally they are able to selectively compete with pathogenic bacteria by production of antimicrobial substances with specific efficacy and are ideally able to simultaneously enhance the growth and viability of beneficial microflora, in particular in the gut or in the gills. Further, they are preferably able to stimulate a systemic immune response in the treated animals.

The mutants or variants according to the invention are preferably spontaneous mutants. The term “spontaneous mutant” refers to mutants or variants that arise from the parent strain without the intentional use of mutagens. I.e. they are considered as not genetically modified (non-GMO). Such spontaneous mutants may be obtained by classical methods, such as growing the Bacillus subtilis strain in the presence of UV light and/or by applying high temperature or protoplast formation and/or in the presence of a certain antibiotic to which the parent strain is susceptible and testing any resistant mutants for improved biological activity or improved ability to enhance one or more of the indicia of animal health, in particular gut health. Other methods for identifying spontaneous mutants are known to those of ordinary skill in the art. But besides these preferred spontaneous mutants all other kinds of mutants or variants, like mutants obtained by genetic engineering, are also comprised by the invention.

One particular embodiment of the invention are naturally non-occurring mutants, in particular spontaneous mutants as defined before, of the bacteria of the invention, in particular of the strains DSM 33349, DSM 33350, DSM 33351 or DSM 33352, characterized by the features as mentioned above.

In a preferred embodiment of the invention, the mutants and variants of the strains DSM 33349, DSM 33350, DSM 33351 or DSM 33352 have the same identifying characteristics like the parent strain from which they were derived.

In a preferred embodiment of the invention, the bacteria, preparations and compositions of the present invention are administered orally to animals or human beings.

Thus, a further subject of the invention are compositions, such as feedstuffs, foodstuffs, drinking and rearing water as well as pharmaceutical compositions, in particular therapeutic compositions, containing at least one bacterium, in particular at least one Bacillus strain, preferably B. subtilis strain, of the invention and/or at least one preparation of the invention and/or at least one composition of the invention as mentioned before.

A further subject of the invention is also the use of at least one bacterium, in particular at least one Bacillus strain, preferably B. subtilis strain, and/or at least one preparation and/or at least one composition of the invention as a probiotic ingredient (DFM) in feed or food products.

In particular, mixtures or combinations of at least two or three strains of the invention may be used, very preferred mixtures or combinations of at least two or at least three strains selected from the strains DSM 33349, DSM 33350, DSM 33351 and DSM 33352.

Preferred foodstuffs according to the invention are dairy products, in particular yoghurt, cheese, milk, butter and quark.

The cells of the bacteria of the invention may be present, in particular in the compositions of the invention, as spores (which are dormant), as vegetative cells (which are growing), as transition state cells (which are transitioning from growth phase to sporulation phase) or as a combination of at least two, in particular all of these types of cells. In a preferred embodiment, the composition of the invention comprises mainly spores, wherein preferably at least 80% or at least 90% of the Bacillus cells are spores, or only spores, i.e. all Bacillus cells as contained in the composition are spores.

In addition or as alternative the cells of the bacteria may also be used in non-living, inactivated form, as also the non-living cells are expected to still have a probiotic effect. Ways to inactivate the cells are known to those skilled in the art.

The bacteria of the invention, when administered to animals, preferably enhance the health of such animals and/or improve the general physical condition of such animals and/or improve the feed conversion rate of such animals and/or decrease the mortality rate of such animals and/or increase the survival rate of such animals and/or improve the weight gain of such animals and/or increase the productivity of such animals and/or increase the disease resistance of such animals and/or modulate the immune response of such animals and/or establish or maintain a healthy gut microflora in such animals and/or improve the meat quality of such animals, in particular the meat elasticity and/or the meat hardness, and/or reduce the pathogen shedding through the feces of such animals. In particular the strains and compositions of the invention might be used to assist in re-establishing a healthy balance of the gut or gill microflora after administration of antibiotics for therapeutic purposes.

A further subject of the invention is therefore a method of enhancing the health of animals and/or of improving the general physical condition of animals and/or of improving the feed conversion rate of animals and/or of decreasing the mortality rate of animals and/or of increasing the survival rates of animals and/or of improving the weight gain of animals and/or of increasing the productivity of animals and/or of increasing the disease resistance of animals and/or of modulating the immune response of animals and/or of establishing or maintaining a healthy gut microflora in animals and/or of improving the meat quality of animals and/or of reducing the pathogen shedding through the feces of animals, wherein at least one strain and/or at least one preparation and/or at least one composition of the invention is administered to animals, in particular to aquatic animals, preferably in aquaculture.

A further subject of the invention is therefore also the use of at least one strain and/or at least one preparation and/or at least one composition of the invention for enhancing the health of animals and/or for improving the general physical condition of animals and/or for improving the feed conversion rate of animals and/or for decreasing the mortality rate of animals and/or for increasing the survival rates of animals and/or for improving the weight gain of animals and/or for increasing the productivity of animals and/or for increasing the disease resistance of animals and/or for modulating the immune response of animals and/or for establishing or maintaining a healthy gut microflora in animals and/or for improving the meat quality of animals and/or for reducing the pathogen shedding through the feces of animals, wherein the at least one strain and/or at least one preparation and/or at least one composition of the invention is administered to animals, in particular to aquatic animals, preferably in aquaculture.

A further subject of the invention is therefore also at least one strain and/or at least one preparation and/or at least one composition of the invention, for enhancing the health of animals and/or for improving the general physical condition of animals and/or for improving the feed conversion rate of animals and/or for decreasing the mortality rate of animals and/or for increasing the survival rate of animals and/or for improving the weight gain of animals and/or for increasing the productivity of animals and/or for increasing the disease resistance of animals and/or for modulating the immune response of animals and/or for establishing or maintaining a healthy gut microflora in animals and/or for improving the meat quality of animals and/or for reducing the pathogen shedding through the feces of animals, wherein the animals are preferably aquatic animals, in particular aquatic animals reared in aquaculture.

“Increasing the productivity of animals” refers in particular to any of the following: production of more or higher quality eggs, milk or meat or increased production of weaned offspring.

The methods and uses of the strains, preparations and compositions of the invention can be therapeutic or non-therapeutic. In a particularly preferred embodiment of the invention, the methods and uses are non-pharmaceutic, in particular feeding applications.

As the untreated manure of animals due to pathogenic bacteria and other ingredients may have a detrimental environmental effect, in particular with respect to the animals themselves and/or with respect to human beings getting in contact with the manure, which can be avoided by either feeding the animals or directly treating the manure or the bedding of the animals with the bacteria, compositions or preparations of the invention, therefore a further subject of the invention is a method of controlling and/or avoiding detrimental environmental effects of manure or contaminated liquids, the method comprising the step of applying to manure, contaminated liquids, litter, a pit, or a manure pond at least one strain, at least one preparation and/or at least one composition according to the invention. Preferably the composition is applied in liquid form, for example by spraying, or as a powder, for example by strewing.

As detrimental bacteria may have a negative influence on the consistency of litter and in particular may effect a rather fluid or highly fluid litter, which might lead to foot pad lesions of poultry and which can be avoided by feeding the animals with the strains, compositions or preparations of the invention, therefore a further subject of the invention is a method of controlling and/or improving the consistency of litter, in particular a method of ensuring a solid consistency of litter and/or a method of avoiding foot pad lesions, the method comprising the step of feeding animals, in particular poultry, with at least one strain, at least one preparation and/or at least one composition according to the invention.

The bacteria, preparations and compositions of the invention are very preferably used for improving the quality of water or aqueous solutions. A further subject of the invention is therefore also a method of controlling and/or improving the quality of water or aqueous solutions, in particular of drinking water and/or rearing water and/or effluent water and/or wastewater, comprising the step of applying to water or an aqueous solution at least one strain and/or at least one preparation and/or at least one composition of the invention.

Inorganic nitrogen compounds do not only represent a problem in water and aqueous solutions of aquaculture facilities, but for example also in different kinds of effluent water and wastewater, in particular in wastewater treatment facilities. Treatments of soils leads often to a eutrophication of the water which is enriched with inorganic nitrogen compounds. Treatment of the effluent water or wastewater with the strains and/or preparations and/or compositions according to the invention can help to remedy this problem.

Further, the bacteria, preparations and compositions of the invention can also be used for treating microbial diseases of plants. A further subject of the invention is therefore also a method of treating and/or preventing microbial diseases of plants, in particular of cultivated plants, comprising the step of applying to the plants at least one strain and/or at least one preparation and/or at least one composition of the invention. The application may be carried out in liquid form, such as by spraying, or in solid form, in particular in form of a powder, preferably as a formulated powder.

By using the strains, preparations and compositions of the invention preferably an improvement of at least one of the features as mentioned before is realized, wherein realization of the feature preferably means an improvement of at least 1%, more preferably of at least 3 or at least 5%, in comparison to an adequate negative control. As negative control averages known in the animal husbandry field may be used, but preferably as negative control animals which are subjected to the same treatment like the animals tested are used, but without administration of the strains and/or preparations and/or compositions of the invention.

In particular, the strains, preparations and compositions of the invention may be administered or fed to an animal in an amount effective to inhibit and/or decrease the growth of pathogenic bacteria, viruses and protozoa, in particular in the animal gut or in the gills of aquatic animals. Such pathogenic bacteria include Clostridia, Listeria, Salmonella, Enterococci, Staphylococci, Aeromonas, Streptococci, Campylobacter, Escherichia coli, Shigella, Haemophilus, Brachyspira, Flavobacterium, Serratia, Yersinia, Edwardsiella, Rennibacterium, Pasteurella and Vibrio. Relatedly, the methods of the present invention may be used to decrease the amount of pathogenic bacteria, viruses and protozoans shed in animal feces. The methods of the present invention may also be used to maintain or increase the growth of beneficial bacteria, such as lactic acid bacteria, in particular in the animal gut or in the gills of aquatic animals. By decreasing pathogenic bacteria and/or increasing or maintaining beneficial bacteria, the compositions of the present invention are able to maintain an overall healthy microflora, in particular in the gut, on the skin or in the gills of aquatic animals.

Thus, a further subject of the invention is also a method of inhibiting and/or decreasing the growth of pathogenic bacteria and/or for maintaining and/or increasing the growth of beneficial bacteria, in particular in an animal gut, on an animal skin or in the gills of aquatic animals, wherein the strains, preparations and compositions of the invention are used and wherein the pathogenic bacteria are preferably selected from Clostridia, in particular from C. perfringens, C. difficile, C. novyi, C. septicum and C. colinum, from Listeria, in particular from L. monocytogenes, L. seeligeri and L. welshimeri, from Salmonella, in particular S. enterica including the subspecies enterica, arizonae, bongori and in particular the serovars, S. gallinarum, S. pullorum, S. typhimurium, S. enteritidis, S. cholerasuis, S. heidelberg and S. infantis, from Enterococci, in particular E. faecalis, E. faecium and E. cecorum, from Staphylococci, in particular S. aureus, from Aeromonas, in particular from A. hydrophila and A. salmonocida, from Streptococci, in particular S. suis, S. gallinaceus and S. agalactiae, from Campylobacter, in particular C. jejuni and C. coli, from Escherichia coli, from Haemophilus, in particular Haemophilus parasuis, from Brachyspira, in particular Brachyspira hyodysenteriae, from Flavobacterium, in particular Flavobacterium columnare, from Serratia, in particular S. liquefaciens, from Yersinia, in particular Y. ruckeri, from Edwardsiella, in particular E. tarda and E. ictaluria, from Rennibacterium, in particular R. salmoninarum, from Pasteurella, in particular P. piscicida, and from Vibrio, in particular V. parahemolyticus, V. harveyi, V. anguillarum, V. ordalii, V. alginolyticus, V. fischeri and V. salmonicida, and the beneficial bacteria are preferably selected from lactic acid bacteria, in particular from lactobacilli and bifidobacteria. Very preferably the inhibited strains are selected from pathogenic strains of aquatic animals, in particular from Streptococcus, in particular S. agalactiae, Vibrio, in particular V. parahemolyticus, V. harveyi, V. anguillarum, V. ordalii, V. alginolyticus, V. fischeri and V. salmonicida, Flavobacterium, in particular Flavobacterium columnare, and Aeromonas, in particular A. hydrophila and A. salmonocida.

In a preferred embodiment of the invention the amount of at least one pathogenic bacterium of aquatic animals, in particular the amount of Streptococcus agalactiae, in particular of S. agalactiae DSM 2134, Vibrio harveyi, in particular V. harveyi DSM 19623, Vibrio parahaemolyticus, in particular V. parahaemolyticus DSM 10027, and/or Aeromonas hydrophila, in particular A. hydrophila DSM 30187, is significantly reduced, preferably by at least 0.5 log, more preferably by at least 1 log, 2 log, or 3 log, wherein reduction of Streptococcus agalactiae is particularly preferred.

Thus, a further subject of the invention are also the strains, preparations and compositions of the invention for inhibiting and/or decreasing the growth of pathogenic bacteria and/or for maintaining and/or increasing the growth of beneficial bacteria in an animal gut or on an animal skin or in the gills of aquatic animals, wherein the pathogenic bacteria are preferably selected from Clostridia, in particular from C. perfringens, C. difficile, C. novyi, C. septicum and C. colinum, from Listeria, in particular from L. monocytogenes, L. seeligeri and L. welshimeri, from Salmonella, in particular S. enterica including the subspecies enterica, arizonae, bongori and in particular the serovars, S. gallinarum, S. pullorum, S. typhimurium, S. enteritidis, S. cholerasuis, S. heidelberg and S. infantis, from Enterococci, in particular E. faecalis, E. faecium and E. cecorum, from Staphylococci, in particular S. aureus, from Aeromonas, in particular from A. hydrophila and A. salmonocida, from Streptococci, in particular S. suis, S. gallinaceus and S. agalactiae, from Campylobacter, in particular C. jejuni and C. coli, from Escherichia coli, from Haemophilus, in particular Haemophilus parasuis, from Brachyspira, in particular Brachyspira hyodysenteriae, from Flavobacterium, in particular F. columnare, from Serratia, in particular S. liquefaciens, from Yersinia, in particular Y. ruckeri, from Edwardsiella, in particular E. tarda and E. ictaluria, from Rennibacterium, in particular R. salmoninarum, from Pasteurella, in particular P. piscicida, and from Vibrio, in particular V. parahemolyticus, V. harveyi, V. anguillarum, V. ordalii, V. alginolyticus, V. fischeri and V. salmonicida, and the beneficial bacteria are preferably selected from lactic acid bacteria, in particular from lactobacilli and bifidobacteria.

Besides or in addition to pathogenic bacteria also other pathogens, in particular viruses and algae, may be inhibited.

In a particular embodiment of the invention, the strains and/or preparations and/or compositions of the invention are able to inhibit at least one virus selected from White Spot Syndrome Virus (WSSv), Taura Syndrome Virus (TSV), Yellow Head Virus (YHV), viruses causing infectious hypodermal and hematopoietic necrosis (IHHN) and IHHNV, virus causing run-deformity syndrome or RDS of Penaeus vannamei, Baculo-like viruses, Infectious Pancreatic Necrosis Virus (IPNV), Hirame rhabdovirus (HIRRV), the Yellowtail Ascites Virus (YAV), Striped Jack Nervous Necrosis Virus (SJNNV), Iridovirus, Infectious salmon anemia (USA) virus, viruses causing Pancreas Disease (PD) or Viral Hemorrhagic Septicemia (VHS), viruses causing viral hemorrhagic septicemia, infectious pancreatic necrosis or viremia of carp, channel catfish virus, grass carp hemorrhagic virus, nodaviridae such as nervous necrosis virus, infectious salmon anemia virus.

In a further particular embodiment of the invention, the strains and/or preparations and/or compositions of the invention are able to inhibit at least one microalga, protozoa or toxin selected from the parasites Ceratomyxa shasta, lchthyophthirius multifillius, Cryptobia salmositica, Lepeophtherius salmonis, Tetrahymena, Trichodina and Epistylus, and dinoflagellate toxins including toxins causing Diaarhetic Shellfish Poisoning (DSP), Paralytic Shellfish Poisoning (PSP), Neurotoxin poisoning (NSP) and Ciguatera.

The occurrence and/or increased growth of pathogens does or can lead to the outbreak of certain diseases.

Vibrios are known to be associated with disease and high mortality in shrimps, but can also infect finfish. Tilapia infections can be associated with Streptococcus agalactiae, a widely distributed bacterium, causing e.g. hemorrhage or erratic swimming. Columnaris disease is caused by Flavobacterium columnare affecting fresh water fish. Salmon and trout farms, for instance, report high annual losses.

Further, the occurrence and/or increased growth of Clostridium perfringens can lead to the outbreak of gut diseases, in particular to the outbreak of necrotic enteritis in swine and poultry. The occurrence and/or increased growth of C. perfringens can also lead to the outbreak of further diseases like bacterial enteritis, gangrenous dermatitis and cholangiohepatitis. Even the mildest form of infection by C. perfringens can already be accompanied by diarrhea, which results in wet litter and by that may lead to secondary diseases like foot pad dermatitis. While C. perfringens type C generally is considered to be the primary cause of necrotic enteritis and necrohemorrhagic enteritis in piglets, type A has been linked to enteric disease in suckling and feeding pigs with mild necrotic enterocolitis and villous atrophy.

Clostridium difficile is an important emerging pathogen that causes diarrhea primarily in neonatal swine. Affected piglets may have dyspnea, abdominal distention, and scrotal edema.

E. cecorum is known to cause lameness, arthritis and osteomyelitis in broilers usually caused by an inflammation of a joint and/or bone tissue. Further E. cecorum can cause an inflammation of the pericardium.

S. gallinaceus can cause septicaemia in poultry. The gross lesions included splenomegaly, hepatomegaly, renomegaly and congestion. Multiple areas of necrosis and/or infarction in the liver and spleen associated with valvular endocarditis were also observed.

C. coli is a foodborne bacterium, most people usually get infected by eating pig meat that contained the bacteria. It causes gastroenteritis and acute enterocolitis in humans, and also of acute diarrheal illnesses. Pigs are the main host, but it can also infect humans, avian species and a wide range of other animals.

Infection with Campylobacter jejuni is one of the most common causes of gastroenteritis worldwide [Acheson et al. 2001]. Infection results from the ingestion of contaminated food or water. C. jejuni is commonly associated with poultry, and it naturally colonises the digestive tract of many bird species [Colles et al. 2009] (20 to 100% of retail chickens are contaminated) as well as cattle especially calves. Some strains of C. jejuni have been reported to cause enteritis and death in newly hatched chicks and poults.

S. suis is an important pathogen in pigs and one of the most important causes of bacterial mortality in piglets after weaning causing septicemia, meningitis and many other infections.

Pathogens can cause further diseases like polyarthritis, fibrinous polyserositis, post-weaning enteric disorders like post-weaning diarrhea and edema disease and swine dysentery.

A further subject of the invention is therefore also a therapeutic composition comprising at least one strain and/or at least one preparation and/or at least one composition of the invention.

A very preferred subject of the invention is therefore a therapeutic composition for treatment and/or prevention of a disease of an aquatic animal, in particular of finfish, preferably tilapia, or of crustaceans, preferably shrimps, and in particular selected from hemorrhage, erratic swimming, Columnaris disease, White Spot Syndrome, and/or of a disease of the gills, comprising at least one strain and/or at least one preparation and/or at least one composition of the invention.

A further preferred subject in this context is therefore a therapeutic composition for treatment and/or prevention of necrotic enteritis and necrohemorrhagic enteritis, in particular sub-clinical necrotic enteritis and necrohemorrhagic enteritis, in animals, preferably swine or poultry, comprising at least one strain and/or at least one preparation and/or at least one composition of the invention.

Another preferred subject in this context is therefore a therapeutic composition for treatment and/or prevention of bacterial enteritis, gangrenous dermatitis, cholangiohepatitis, clostridiosis, diarrhea, dyspnea, abdominal distention, scrotal edema, bumblefoot, foot pad dermatitis, streptococcal mastitis, lameness, arthritis, polyarthritis, fibrinous polyserositis, post-weaning enteric disorders like post-weaning diarrhea and edema disease, dysentery, osteomyelitis, inflammation of joints and/or bone tissue, inflammation of the pericardium, splenomegaly, hepatomegaly, renomegaly, congestion, necrosis, infarction in the liver or spleen, valvular endocarditis, septicemia and/or meningitis, in animals, preferably in swine or poultry, comprising at least one strain and/or at least one preparation and/or at least one composition of the invention.

A further preferred subject of the invention is therefore also the treatment and/or prevention of a disease of an aquatic animal, in particular of finfish, preferably tilapia, or crustaceans, preferably shrimps, and in particular selected from hemorrhage, erratic swimming and Columnaris disease, wherein at least one strain and/or at least one preparation and/or at least one composition of the invention is administered to an aquatic animal in need thereof.

A further subject of the invention is therefore also the treatment and/or prevention of a disease, in particular of a gut disease, preferably of necrotic enteritis or necrohemorrhagic enteritis, in particular of sub-clinical necrotic enteritis or sub-clinical necrohemorrhagic enteritis, in swine or poultry, wherein at least one strain and/or at least one preparation and/or at least one composition of the invention is administered to an animal in need thereof.

A further subject of the invention is therefore also the treatment and/or prevention of a disease, preferably a disease of swine or poultry, selected from bacterial enteritis, gangrenous dermatitis, cholangiohepatitis, clostridiosis, diarrhea, dyspnea, abdominal distention, scrotal edema, bumblefoot, foot pad dermatitis, streptococcal mastitis, lameness, arthritis, polyarthritis, fibrinous polyserositis, post-weaning enteric disorders like post-weaning diarrhea and edema disease, dysentery, osteomyelitis, inflammation of joints and/or bone tissue, inflammation of the pericardium, splenomegaly, hepatomegaly, renomegaly, congestion, necrosis, infarction in the liver or spleen, valvular endocarditis, septicemia and/or meningitis, wherein at least one strain and/or and least one preparation and/or at least one composition of the invention is administered to an animal in need thereof.

The strains and/or preparations and/or compositions of the invention can be administered to animals in feed and/or drinking water and/or rearing water over multiple days throughout the animal's life or during particular stages or portions of the animal's life. For example, the strains and/or preparations and/or compositions can be administered only in a starter diet or only in a finisher diet of farm animals.

A particular subject of the invention is also a method of enhancing the health of human beings and/or of improving the general physical condition of human beings and/or of increasing the disease resistance of human beings and/or of modulating the immune response of human beings and/or of establishing or maintaining a healthy gut microflora in human beings, wherein the strains and/or preparations of the invention or the compositions of the invention, which comprise such strain(s), are administered to human beings.

A further subject of the invention is therefore also the use of strains and/or preparations and/or compositions of the invention for enhancing the health of human beings and/or for improving the general physical condition of human beings and/or for increasing the disease resistance of human beings and/or for modulating the immune response of human beings and/or for establishing or maintaining a healthy gut microflora in human beings, wherein the strains and/or preparations of the invention or the compositions of the invention, which comprise such strain(s), are administered to human beings.

The compositions of the present invention, in particular the feed, food and pharmaceutical compositions as well as the drinking water, rearing water, effluent water or wastewater, preferably comprise the strains of the invention and are administered to animals at a rate of about 1×10³ to about 2×10¹² CFU/g feed or ml water, in particular in a rate of about 1×10³ or about 1×10⁴ or about 1×10⁵ or about 1×10⁶ or about 1×10⁷ or about 1×10⁸ or about 1×10⁹ or about 1×10¹⁰ or about 1×10¹¹ or about 1×10¹² CFU/g feed or ml water, preferably in an amount of about 1×10⁴ to about 1×10¹⁰ CFU/g feed or ml water, more preferably in an amount of 1×10⁴ to 1×10⁷ CFU/g feed or ml water.

Correspondingly, preferred amounts of the strains and/or preparations of the invention in the feed, food and water compositions of the invention range from 0.1 wt.-% to 10 wt.-%, more preferably from 0.2 wt.-% to 5 wt.-%, in particular from 0.3 wt.-% to 3 wt.-%.

The methods of the present invention may be used for all kind of animals, in particular all kind of non-human and non-insect animals, more preferably all kind of vertebrates such as mammals, aquatic animals and birds, with aquatic animals being particularly preferred.

Animals that may benefit from the invention include but are not limited to farm animals, pets, exotic animals, zoo animals, animals used for sports, recreation or work.

Pets are preferably selected from dogs, cats, domestic birds and domestic exotic animals.

The aquatic animals according to the invention are preferably finfish, in particular of the class Actinopterygii, or crustaceans. Actinopterygii include, in particular, tilapia and other cichlids, carps and other cyprinids, salmons and other salmonids, catfish, in particular African catfish and pangasius, tuna, perch, cod, smelt, milkfish, gourami, seabass, in particular barramundi, seabream, grouper and snakehead fish.

Preferred types of salmon and salmonids in this context are the Atlantic salmon, red salmon (sockeye salmon), masu salmon (cherry salmon), king salmon (Chinook salmon), keta salmon (chum salmon), coho salmon, Danube salmon, Pacific salmon, pink salmon and trout. The aquatic animals according to the invention are very preferred tilapia.

Crustaceans include in particular shrimps, lobster, crabs, prawns and crayfish.

Preferred types of shrimps in this context are Litopenaeus, Farfantepenaeus and Penaeus, in particular Penaeus stylirostris, Penaeus vannamei, Penaeus monodon, Penaeus chinensis, Penaeus occidentalis, Penaeus calif orniensis, Penaeus semisulcatus, Penaeus esculentu, Penaeus setiferus, Penaeus japonicus, Penaeus aztecus, Penaeus duorarum, Penaeus indicus, and Penaeus merguiensis. Very preferred according to the invention is the shrimp Penaeus vannamei, also called whiteleg shrimp.

The aquatic animals, and in particular the shrimp, which are treated or fed with the strains and/or preparations and/or compositions according to the invention can in particular be larvae, post-larvae or juvenile shrimp.

The aquatic animals may in particular also be fish which is subsequently processed into fish meal or fish oil. In this connection, the fish are preferably herring, pollack, cod or small pelagic fish like anchovy, blue whiting, capelin, driftfish, jack, mackerel, menhaden, sardine or scad fish. The fish meal or fish oil thus obtained, in turn, can be used in aquaculture for farming edible fish or crustaceans.

The aquatic animals may further be oysters, clams, cockles, arkshells, bivalves, mussels or scallops.

However, the aquatic animals may also be small organisms which are used as feedstuff in aquaculture. These small organisms may take the form of, for example, nematodes, small crustaceans or rotifers.

The farming of aquatic animals may take place in ponds, tanks, basins or else in segregated areas in the sea or in lakes or in rivers, in particular in this case in cages or net pens. Farming may be used for farming the finished edible fish, but also may be used for farming fry which are subsequently released so as to restock the wild fish stocks.

In salmon farming, the fish are preferably first grown into smolts in freshwater tanks or artificial watercourses and then grown on in cages or net pens which float in the sea, ponds or rivers and which are preferably anchored in bays or fjords.

Accordingly, the feedstuff according to the invention is preferably a feedstuff for use in the farming of the above-mentioned animals.

In accordance with the invention, the strains, preparations and compositions of the invention can be employed in a wide dosage range. Daily doses are, for example, in the range of between approximately 1 mg and approximately 500 mg, in particular in the range of approximately 5 mg and approximately 200 mg, in the range of from approximately 10 mg to approximately 100 mg or in the range of from approximately 20 to approximately 60 mg per kilogram live weight.

In a further embodiment of the invention, the animals are farm animals, which are raised for consumption or as food-producers, such as poultry, swine and ruminants.

The poultry may be selected from productive or domestic poultry, but also from fancy poultry or wild fowl. Preferred productive poultry in this context are chickens, turkeys, ducks and geese. The productive livestock in this context is preferably poultry optimized for producing young stock or poultry optimized for bearing meat. Preferred fancy poultry or wild fowl are peacocks, pheasants, partridges, chukkars, guinea fowl, quails, capercaillies, grouse, pigeons and swans, with quails being especially preferred. Further preferred poultry are ratites, in particular ostriches and emus, as well as parrots.

Ruminants according to the invention are preferably selected from cattle, goat and sheep. In one embodiment, the compositions of this invention may be fed to preruminants to enhance their health and, in particular, to decrease the incidence of diarrhea in these animals. Preruminants are ruminants, including calves, ranging in age from birth to about twelve weeks.

The compositions of the invention may comprise at least one carrier or typical feed ingredients or combinations thereof.

Suitable carriers are inert formulation ingredients added to improve recovery, efficacy, or physical properties and/or to aid in packaging and administration. Such carriers may be added individually or in combination. These carriers may be selected from anti-caking agents, anti-oxidation agents, bulking agents, binders, structurants, coatings and/or protectants. Examples of useful carriers include polysaccharides (in particular starches, maltodextrins, methylcelluloses, gums, chitosan and/or inulins), protein sources (in particular skim-milk powder and/or sweet-whey powder), peptides, sugars (in particular lactose, trehalose, sucrose and/or dextrose), lipids (in particular lecithin, vegetable oils and/or mineral oils), salts (in particular sodium chloride, sodium carbonate, calcium carbonate, chalk, limestone, magnesium carbonate, sodium phosphate, calcium phosphate, magnesium phosphate and/or sodium citrate), and silicates (in particular clays, in particular beolite clay, amorphous silica, fumed/precipitated silicas, zeolites, Fuller's earth, Baylith®, clintpolite, montmorillonite, diatomaceous earth, talc, bentonites, and/or silicate salts like aluminium, magnesium and/or calcium silicate). Suitable carriers for animal feed additives are set forth in the American Feed Control Officials, Inc.'s Official Publication, which publishes annually. See, for example Official Publication of American Feed Control Officials, Sharon Krebs, editor, 2006 edition, ISBN 1-878341-18-9. The carriers can be added after concentrating the fermentation broth and/or during and/or after drying. Preferred carriers according to the invention are selected from calcium carbonate, diatomaceous earth and vegetable oil.

A preferred embodiment of the invention are concentrate compositions, in particular feed additive compositions, i.e. compositions suitable for preparing a feed composition, which comprise at least one strain of the invention and at least one carrier as mentioned before, wherein the at least one strain is preferably comprised in an amount of 0.1 to 10 wt.-%, more preferably in an amount of 0.2 to 5 wt.-%, in particular in an amount of 0.3 to 3 wt.-%, above all in an amount of 0.4 to 2.2 wt.-%, and the at least one carrier is preferably comprised in an amount of at least 90 wt.-%, preferably in an amount of 90 to 99.9 wt.-%, more preferably in an amount of 95 to 99.8 wt.-%, in particular in an amount of 97 to 99.7 wt.-%, above all in an amount of 97.8 to 99.6 wt.-%, and wherein the carrier consists preferably substantially of limestone, in particular of limestone with smaller parts of diatomaceous earth and/or vegetable oil.

These preferred compositions of the invention, which contain stabilized strains, can be used for the preparation of feed and pharmaceutical compositions as well as drinking and rearing water which preferably comprise the strains according to the invention in an amount as mentioned in the specification above. In a preferred embodiment 50 to 1000 grams of such a concentrate composition, in particular 50, 100, 250, 500 or 1000 grams of such a concentrate composition, are used per ton of feed, drinking or rearing water to provide compositions which can be used for feeding animals. These concentrate compositions preferably comprise at least one strain of the invention in an amount of 1×10⁹ to 2×10¹¹ CFU, in particular 2×10⁹ to 1×10¹¹ CFU, per g of the concentrate composition.

Starting from these concentrate compositions, feed and food compositions can be prepared by mixing the concentrate compositions with typical feed or food ingredients, respectively.

Suitable typical animal feed ingredients which may be contained in the compositions according to the invention and/or used in the preparation of feed compositions starting from concentrate compositions according to the invention include one or more of the following: proteins, carbohydrates, fats, further probiotics and/or further inorganic nitrogen compounds degrading microorganisms, prebiotics, enzymes, vitamins, immune modulators, milk replacers, minerals, amino acids, carriers, coccidiostats, acid-based products and/or medicines, such as antibiotics.

Carbohydrates containing components which may be used according to the invention are for example forage, roughage, wheat meal, corn meal, sunflower meal or soya meal, and mixtures thereof.

Proteins containing components which may be used according to the invention are for example soya protein, pea protein, wheat gluten, corn gluten, rice, canola meal, meal of marine animals, in particular fishmeal, meal of terrestrial animals, and mixtures thereof. “Meal of marine animals” includes meat meal, meat and bone meal, blood meal, liver meal, poultry meal, silkworm meal, silkworm pupae meal and combinations thereof.

Fats are typically provided as oils of marine animals, vegetable oils or oils of microorganisms, in particular oils of microalgae, or combinations thereof. Examples of vegetable oils are soybean oil, rapeseed oil, sunflower seed oil, canola oil, cottonseed oil, flaxseed oil and palm oil. Oils of marine animals include fish oil as well as oil of krill, bivalves, squids or shrimps and further fatty oils from fish of the families Engraulidae, Carangidae, Clupeidae, Osmeridae, Scombridae and/or Ammodytidae. Examples of oils of microalgae are in particular oil of Labyrinthulea, preferably oil of Schizochytria or Thraustochytria. Besides the isolated oils the defatted biomass itself may also be used as fat source, i.e. in particular the meal of a marine animals, preferably fishmeal, or a plant meal, in particular soybean meal, rapeseed meal, sunflower meal, canola meal, cottonseed meal and/or flax seed meal.

Proteins containing components which additionally contain fats which may be used according to the invention are for example fish meal, krill meal, bivalve meal, squid meal or shrimp shells, as well as combinations thereof.

The feedstuff according to the invention has preferably a total protein content of 20 to 50 wt.-% and/or a total fat content of 1 to 15 wt.-% and/or a total carbohydrate content of 20 to 60 wt.-%.

Further probiotics (DFM) and/or microorganisms which may be used according to the invention in combination with the strains and preparations of the invention are preferably bacteria selected from the species Bacillus subtilis, Bacillus licheniformis, Bacillus lentus, Bacillus pumilus, Bacillus laterosporus, Bacillus coagulans, Bacillus alevi, Bacillus cereus, Bacillus badius, Bacillus thurigiensis, Enterococcus faecium, and Pediococcus acidilactici. Preferred examples are Bacillus pumilus DSM 32539 and Bacillus subtilis DSM 32540 (both deposited with the DSMZ on Jun. 14, 2017 under the provisions of the Budapest Treaty on the International Recognition of the Deposit of Microorganisms for the Purpose of Patent Procedure) and derivatives thereof, Bacillus licheniformis DSM 32314 and Bacillus subtilis DSM 32315 (both deposited with the DSMZ on May 12, 2016 under the provisions of the Budapest Treaty on the International Recognition of the Deposit of Microorganisms for the Purpose of Patent Procedure) and derivatives thereof, Bacillus subtilis PB6 (as described in U.S. Pat. No. 7,247,299 and deposited as ATCC Accession No. PTA-6737), which is sold by Kemin under the trademark CLOSTAT®, Bacillus subtilis C-3102 (as described in U.S. Pat. No. 4,919,936 and deposited as FERM BP-1096 with the Fermentation Research Institute, Agency of Industrial Science and Technology, in Japan), sold by Calpis as CALSPORIN®, Bacillus subtilis DSM 17299, as sold by Chr. Hansen under the trademark GalliPro®, Bacillus licheniformis DSM 17236, as sold by Chr. Hansen under the trademark GalliProTect®, a mixture of Bacillus licheniformis DSMZ 5749 and Bacillus subtilis DSMZ 5750 spores, as sold by Chr. Hansen under the trademark BioPlus®YC, B. subtilis DSM 29784, as sold by Adisseo/Novozymes under the trademark Alterion®, Bacillus subtilis, as sold by Chr. Hansen under the trademark PORCBOOST®, or Bacillus coagulans strains as described in U.S. Pat. No. 6,849,256. Other non-Bacillus probiotics, such as Saccharomyces cerevisiae, Pichia pastoris, Aspergillus niger, Aspergillus oryzae, or Hansenula, may also be used in compositions of the present invention. In particular in food compositions further probiotics which are known to be useful to the human health may be used such as lactic acid producing bacteria, in particular lactobacilli, or Bifidobacteria. If said further probiotics are not formulated as part of the compositions of the present invention, they may be administered together (either at the same time or at different times) with the compositions of the present invention.

Prebiotics which may be used according to the invention are preferably oligosaccharides, in particular selected from galactooligosaccharides, sialyloligosaccharides, lactulose, lactosucrose, fructooligosaccharides, palatinose or isomaltose oligosaccharides, glycosyl sucrose, maltooligosaccharides, isomaltooligosaccharides, cyclodextrins, gentiooligosaccharides, soybean oligosaccharides, xylooligosaccharides, dextrans, pectins, polygalacturonan, rhamnogalacturonan, mannan, hemicellulose, arabinogalactan, arabinan, arabinoxylan, resistant starch, mehbiose, chitosan, agarose, inulin, tagatose, polydextrose, and alginate.

Enzymes which may be used in feed compositions according to the invention and which may aid in the digestion of feed, are preferably selected from phytases (EC 3.1.3.8 or 3.1.3.26), xylanases (EC 3.2.1.8), galactanases (EC 3.2.1.89), galactosidases, in particular alpha-galactosidases (EC 3.2.1.22), proteases (EC 3.4), phospholipases, in particular phospholipases A1 (EC 3.1.1.32), A2 (EC 3.1.1.4), C (EC 3.1.4.3), and D (EC 3.1.4.4), lysophospholipases (EC 3.1.1.5), amylases, in particular alpha-amylases (EC 3.2.1.1); lysozymes (EC 3.2.1.17), glucanases, in particular beta-glucanases (EC 3.2.1.4 or EC 3.2.1.6), glucoamylases, cellulases, pectinases, or any mixture thereof.

Examples of commercially available phytases include Bio-Feed™ Phytase (Novozymes), Ronozyme® P and HiPhos™ (DSM Nutritional Products), Natuphos™ (BASF), Finase® and Quantum® Blue (AB Enzymes), the Phyzyme® XP (Verenium/DuPont) and Axtra® PHY (DuPont). Other preferred phytases include those described in e.g. WO 98/28408, WO 00/43503, and WO 03/066847.

Examples of commercially available xylanases include Ronozyme® WX and G2 (DSM Nutritional Products), Econase® XT and Barley (AB Vista), Xylathin® (Verenium) and Axtra® XB (Xylanase/beta-glucanase, DuPont). Examples of commercially available proteases include Ronozyme® ProAct (DSM Nutritional Products).

Vitamins which may be used according to the invention are for example vitamin A, vitamin D3, vitamin E, vitamin K, e.g., vitamin K3, vitamin B12, biotin, choline, vitamin B1, vitamin B2, vitamin B6, niacin, folic acid and pantothenate, e.g., Ca-D-pantothenate, or combinations thereof.

Immmune modulators which may be used are for example antibodies, cytokines, spray-dried plasma, interleukins, or interferons, or combinations thereof.

Minerals which may be used according to the invention are for example boron, cobalt, chloride, chromium, copper, fluoride, iodine, iron, manganese, molybdenum, selenium, zinc, calcium, phosphorous, magnesium, potassium, or sodium, or combinations thereof.

Amino acids which may be used according to the invention are for example lysine, alanine, threonine, methionine or tryptophan, or combinations thereof.

The feedstuffs of the invention may further comprise betaine and/or choline and/or other physiologically effective methyl group donors. The feedstuffs may further comprise polyunsaturated fatty acids, in particular DHA and/or EPA.

Thus, a further embodiment of the invention is a method of preparing an animal feed composition comprising mixing at least one strain and/or at least one preparation and/or at least one concentrate composition of the invention, in particular in an amount effective to enhance animal health, in particular gut health, with feed ingredients, such as proteins, lipids and/or carbohydrates, and optionally further beneficial substances, preferably as mentioned before, to provide a feeding product. This method may comprise for example also a pelleting step.

Standard pelleting processes known to those of skill in the art may be used, including extrusion processing of dry or semi-moist feeds. Preferred pelleting temperatures are between about 65° C. and about 120° C.

The strains and compositions of the present invention can be obtained by culturing the strains of the invention according to methods well known in the art, including by using the media and other methods as described for example in U.S. Pat. No. 6,060,051, EP0287699 or US2014/0010792. Conventional large-scale microbial culture processes include submerged fermentation, solid state fermentation, or liquid surface culture. Towards the end of fermentation, as nutrients are depleted, the cells of the strains begin the transition from growth phase to sporulation phase, such that the final product of fermentation is largely spores, metabolites and residual fermentation medium. Sporulation is part of the natural life cycle of these strains and is generally initiated by the cell in response to nutrient limitation. Fermentation is configured to obtain high levels of colony forming units of the Bacillus subtilis cells and to promote sporulation. The bacterial cells, spores and metabolites in culture media resulting from fermentation may be used directly or concentrated by conventional industrial methods, such as centrifugation, tangential-flow filtration, depth filtration, and evaporation. The concentrated fermentation broth may be washed, for example via a diafiltration process, to remove residual fermentation broth and metabolites.

The fermentation broth or broth concentrate can be dried with or without the addition of carriers using conventional drying processes or methods such as spray drying, freeze drying, tray drying, fluidized-bed drying, drum drying, or evaporation. The resulting dry products may be further processed, such as by milling or granulation, to achieve a specific particle size or physical format. Carriers, as described above, may also be added post-drying.

Preparations of the strains of the invention may be cell-free preparations or preparations containing cell debris or preparations containing a mixture of intact cells and cell debris.

Cell-free preparations of the strains of the invention can be obtained for example by centrifugation and/or filtration of fermentation broth. Depending on the technique used, these cell-free preparations may not be completely devoid of cells, but may still comprise a smaller amount of cells. As the cells secret compounds like metabolites, enzymes and/or peptides into the surrounding medium, the supernatant of the cells comprises a mixture of such compounds, in particular metabolites, enzymes and/or peptides, as secreted by the cells. Thus, in a preferred embodiment of the invention, the preparation of the strains is a supernatant of the fermentation broth. The preparation of the strains, in particular the supernatant, can also be used in dried form, wherein drying can be carried out for example by spray-drying or freeze-drying.

Compositions comprising cell debris of the strains may be obtained by rupturing the cells applying techniques as known to those of skill in the art, for example by mechanical means or by applying high pressure. Depending on the degree of force applied, a composition comprising only ruptured cells or a composition comprising a mixture of cell debris and intact cells is obtained. Homogenization of the cells may be realized for example by utilizing a French cell press, sonicator, homogenizer, microfluidizer, ball mill, rod mill, pebble mill, bead mill, high pressure grinding roll, vertical shaft impactor, industrial blender, high shear mixer, paddle mixer, and/or polytron homogenizer. Suitable alternatives are enzymatic and/or chemical treatment of the cells.

Cell-free preparations of the invention comprise also preparations which are obtained by first rupturing the cells by applying techniques as mentioned before and subsequently removing the cell debris and the remaining intact cells. Removing of the cell debris and remaining intact cells can be carried out in particular by centrifugation and/or filtration.

The preparations of the strains of the invention may comprise as active compounds at least one metabolite, preferably a mixture of metabolites, as further described below, and/or at least one enzyme selected from proteases, in particular subtilisin, xylanases and/or cellulases, and/or at least one peptide, and/or combinations thereof.

A preparation containing an effective mixture of metabolites as contained in the strains of the invention and/or as contained in the cell preparations as mentioned before, can be obtained for example according to the methods set forth in U.S. Pat. No. 6,060,051. In particular the preparation can be obtained by precipitating the metabolites as contained in the preparations mentioned before by using organic solvents like ethyl acetate and subsequent redissolving of the precipitated metabolites in an appropriate solvent. The metabolites may subsequently be purified by size exclusion filtration that groups metabolites into different fractions based on molecular weight cut-off.

The preparation containing an effective mixture of metabolites of the invention preferably comprises at least five, more preferably at least 6, 7, 8, 9, 10 or 12, in particular all metabolites of the respective strains of the invention. The metabolites possess preferably a molecular weight of between 400 and 5000 Dalton, more preferably of between 800 and 4000 Dalton.

Preferably according to the invention always an effective amount of the strains and/or preparations and/or compositions of the invention is used in the embodiments of the invention. The term “effective amount” refers to an amount which effects at least one beneficial effect to an animal and/or to the environment, in particular with respect to the features as already mentioned before, in comparison to an animal that has not been administered the strains and/or preparations and/or compositions of the invention, but besides that has been administered the same diet (including feed and other compounds).

In case of therapeutic applications preferably a therapeutic amount of the strains and/or preparations and/or compositions of the invention is used. The term “therapeutic amount” refers to an amount sufficient to ameliorate, reverse or prevent a disease state in an animal. Optimal dosage levels for various animals can easily be determined by those skilled in the art, by evaluating, among other things, the composition's ability to (i) inhibit or reduce pathogenic bacteria in the gut or in the gills at various doses, (ii) increase or maintain levels of beneficial bacteria and/or (iii) enhance animal health, in particular gut health, at various doses.

### WORKING EXAMPLES

### Example 1: Capacity of Strains to Reduce Nitrogen

Bacillus strains from diverse environmental samples were screened regarding their water remediation characteristics. Various tests were carried out for determination of the capacity to reduce nitrogen compounds under broad conditions in order to receive superior strains for aquaculture bioremediation. Strains were tested under different salinities (0-3%) and varying C:N-ratios (20-2600). The four B. subtilis strains DSM 33351, DSM 33352, DSM 33349 and DSM 33350 showed very promising properties, which are described in the following.

All nitrite (NO₂—N) measurements were carried out using the Nitrite cuvette test 0.015-0.6 mg/L NO₂—N and Nitrite cuvette test 0.6-6.0 mg/L NO₂—N(Hach Company, Loveland, Colo., USA). All ammonia (NH4₊-N) concentrations were determined by a flow injection analyzer (FIA, applied system: FOSS FIAstar 5000). Within the system sample aliquots were alkalized in order to generate ammonia (NH3) from solvated ammonium ions (NH4+) quantitatively. Ammonia diffused via a semipermeable membrane (gas diffusion). Changes of the pH can then be monitored via an indicator-solution (photometric detection, applied indicator based on 5′,5″-Dibrom-o-kresolsulfonphthalein (“Bromkresolpurpur”, Sigma-Aldrich, St. Louis, Mo., USA). Extinction will increase non-linear with higher ammonia concentrations and needs to be calibrated properly. The method has a validated measuring area of 1-13 mg/L (linear range) and 10-130 mg/L (non-linear range).

All strains were tested for the capacity of ammonia reduction in C minimal medium containing only glucose as carbon source and 5 ppm ammonia as sole nitrogen source under different salinities. For each B. subtilis strain, 50 μl of a glycerol stock was inoculated to 10 ml C minimal medium with a C:N ratio of 68 (0.0352 g/l MnSO₄×1H₂O, 2.46 g/l MnSO₄×7H₂O, 0.2 KH₂PO₄, 0.6 g/l K2HPO₄, 0.02 FeSO₄×7H₂O, 0.2792 g/l (NH₄)₂SO₄, 10 g/l glucose and 0 or 15 or 30 g/l NaCl) and incubated in 100 ml shaking flasks at 28° C. and 200 rpm. After 48 h of incubation, the concentration of ammonia was measured with the method described above. The percentage amount of ammonia reduced by each strain was calculated referring to the initial concentration, which was determined in the non-inoculated control for each salinity (Table 1.1). Strains DSM 33351, DSM 33352, DSM 33349 and DSM 33350 were able to efficiently remove ammonia in C minimal medium containing only glucose as carbon source and 5 ppm ammonia as nitrogen source, as maximum 0.3% of the initial ammonia concentration were still detectable after 48 h under all salinity conditions.

TABLE 1.1
Ammonia reduction in C minimal medium containing
only glucose as carbon source and 5 ppm ammonia
as nitrogen source under different salinities.

| | *Removed ammonia [%]/**Ammonia | | |
|---|---|---|---|
| | concentration [mg/l] | | |
| Strain* | 0% NaCl | 1.5% NaCl | 3.0% NaCl |
| DSM 33351 | 99.7 | 100.0 | 100.0 |
| DSM 33352 | 99.7 | 100.0 | 99.7 |
| DSM 33349 | 99.9 | 100.0 | 99.9 |
| DSM 33350 | 99.7 | 100.0 | 99.7 |
| Initial concentration** | 4.45 | 4.53 | 4.47 |

All strains were further tested for their capacity of nitrite reduction in C minimal medium containing only glucose as carbon source and 5 ppm nitrite as nitrogen source. For each B. subtilis strain, 50 μl of a glycerol stock was inoculated to 10 ml C minimal medium with a C:N ratio of 2600 (0.0352 g/l MnSO₄×1H₂O, 2.46 g/l MnSO₄×7H₂O, 0.2 KH₂PO₄, 0.6 g/l K2HPO₄, 0.02 FeSO₄×7H₂O, 0.0075 g/l NaNO₂, 10 g/l glucose and 0 or 15 or 30 g/l NaCl) and incubated in 100 ml shaking flasks at 28° C. and 200 rpm. After 48 h of incubation, the concentration of nitrite was measured with the method described above. The percentage of nitrite reduced by each strain was calculated referring to the initial concentration, which was determined in the non-inoculated control for each salinity (Table 1.2). Under 0 and 15% salinity, strains DSM 33351, DSM 33352, DSM 33349 and DSM 33350 were able to efficiently remove all nitrite within 48 h. Under 3% salinity, strain DSM 33351 and DSM 33350 reduced nitrite to 0 mg/ml, while strain DSM 33352 and DSM 33349 removed 67 and 87% in reference to 5.15 mg/l as initial concentration respectively.

TABLE 1.2
Nitrite reduction in C minimal medium containing
only glucose as carbon source and 5 ppm nitrite
as nitrogen source under different salinities.

| | *Removed nitrite [%]/**Nitrite | | |
|---|---|---|---|
| | concentration [mg/l] | | |
| Strain* | 0% NaCl | 1.5% NaCl | 3.0% NaCl |
| DSM 33351 | 100.0 | 100.0 | 100.0 |
| DSM 33352 | 100.0 | 100.0 | 67.09 |
| DSM 33349 | 100.0 | 100.0 | 86.64 |
| DSM 33350 | 100.0 | 100.0 | 100.0 |
| Initial concentration** | 5.24 | 5.19 | 5.19 |

All strains were tested for their capacity of ammonia and nitrite reduction in C minimal medium containing only glucose as carbon source and final concentration of 2.5 ppm of both nitrite and ammonia and a C:N ratio of 20. 10 ml of the respective media (0.0352 g/l MnSO₄×1H₂O, 2.46 g/l MnSO₄×7H₂O, 0.2 KH₂PO₄, 0.6 g/l K2HPO₄, 0.02 FeSO₄×7H₂O, 0.1396 g/l (NH4)₂SO₄, 0.00375 g/l NaNO₂, 1.517 g/l glucose and 0 or 15 or 30 g/l NaCl) were inoculated with a single colony of strain DSM 33351, DSM 33352, DSM 33349 or DSM 33350 grown on TSA agar for 24 h at 37° C. Cultures were incubated at 28° C. at 200 rpm. After 48 h, the concentrations of ammonia and nitrite were determined with the above described methods. The amount of ammonia and nitrite reduced by each strain was calculated referring to the initial concentration, which was determined in the non-inoculated control for each salinity (Table 1.3). Strains DSM 33351, DSM 33352, DSM 33349 and DSM 33350 were able to simultaneously remove 99.7% of the initial ammonia and 100% of the initial nitrite after 48 h under all salinities in C minimal medium containing only glucose as carbon source and final concentration of 2.5 ppm nitrite and ammonia as nitrogen source.

TABLE 1.2
Ammonia and nitrite reduction in C minimal medium containing only
glucose as carbon source and final concentration of 2.5 ppm nitrite
and ammonia as nitrogen source under different salinities.

| | *Removed ammonia | | | *Removed nitrite | | |
|---|---|---|---|---|---|---|
| | [%]/**Ammonia | | | [%]/**Nitrite | | |
| | concentration [mg/l] | | | concentration [mg/l] | | |
| | 0% | 1.5% | 3.0% | 0% | 1.5% | 3.0% |
| Strain* | NaCl | NaCl | NaCl | NaCl | NaCl | NaCl |
| DSM 33351 | 99.7 | 99.7 | 99.7 | 100.0 | 100.0 | 100.0 |
| DSM 33352 | 99.7 | 99.7 | 99.7 | 100.0 | 100.0 | 100.0 |
| DSM 33349 | 99.7 | 99.7 | 99.7 | 100.0 | 100.0 | 100.0 |
| DSM 33350 | 99.7 | 99.7 | 99.7 | 100.0 | 100.0 | 100.0 |
| Initial | 2.12 | 2.13 | 2.24 | 2.27 | 2.20 | 2.06 |
| concentration** | | | | | | |

Overall, these data show that B. subtilis strains DSM 33351, DSM 33352, DSM 33349 and DSM 33350 can use both ammonia and nitrite as sole nitrogen source and efficiently remove both compounds within 48 h.

### Example 2: Outgrowth of Spores in C Minimal Medium and Nitrogen Reduction

The ability of Bacillus strains to produces spores is a great advantage for receiving long-term stable product.

In order to produce spores of B. subtilis DSM 33351, DSM 33352, DSM 33349 and DSM 33350, the strains were pre-grown for 14 hours at 30° C. in 50 g of medium containing 43 g/kg soymeal and 22 g/kg of a sugar-solution (320 g/kg glucose, 90 g/kg fructose, 350 g/kg maltose and 100 g/kg maltotriose) and were shaken at 200 rpm. Next, 14 g of the previous culture was used to inoculate 200 g of spore-production-medium and were shaken at 200 rpm while incubated at 30° C. The spore-production-medium consisted of 21 g/kg of the abovementioned sugar-solution, 43 g/kg soymeal, 0.29 g/kg MgSO₄×7 H₂O, 0.032 g/kg MnSO₄×H₂O, 0.023 g/kg FeSO₄×7H₂O and 0.003 g/kg ZnSO₄×7H₂O. After 48 hours of incubation, the spores were harvested by centrifugation and resuspended in 20% glycerol in PBS buffer and stored at −80° C.

Spores DSM 33351, DSM 33352, DSM 33349 and DSM 33350 were tested regarding their potential to grow out in C-minimal medium with ammonia and nitrite as nitrogen source and regarding their capacity to reduce ammonia and nitrite efficiently.

For testing the outgrowth in a microtiter plate based assay, 10⁶ CFU/ml spores of strain DSM 33351, DSM 33352, DSM 33349 and DSM 33350 were inoculated to a final volume of 150 μl ml of the respective C-minimal medium with glucose as only carbon source and with ammonia and nitrite as nitrogen source (0.0352 g/l MnSO₄×1H₂O, 2.46 g/l MnSO₄×7H₂O, 0.2 KH₂PO₄, 0.6 g/l K2HPO₄, 0.02 FeSO₄×7H₂O, 0.1396 g/l (NH₄)2SO₄, 0.00375 g/l NaNO₂ and 10 g/l glucose (C:N ratio=20)) supplemented with 10% PrestoBlue™ (Thermo Fisher Scientific, Waltham, Mass., USA). Spores were incubated at 37° C. and orbital shaking at 350 rpm for 30 h in a microtiter plate reader (TECAN Infinite® M1000 Pro). The detection of outgrowth of the spores occurred via the metabolic activity of the outgrowing cells, which reduce the nonfluorescent resazurin of PrestoBlue™ to bright red fluorescent resorufin. The fluorescence was measured real-time during cultivation (excitation filter: 550-12 nm; emission filter: 590 nm). The resulting kinetics was used to calculate the timepoint of spore outgrowth based on a defined threshold. The hours until outgrowth for strains DSM 33351, DSM 33352, DSM 33349 and DSM 33350 are depicted in Table 2.1. All strains were able to grow out in C minimal medium containing only glucose as C source and final concentration of 2.5 ppm nitrite and ammonia within 23.35 h, while DSM 33349 grew out fastest with 18.89 h.

TABLE 2.1
Outgrowth of spores in C minimal medium containing only
glucose as carbon source and final concentration of
2.5 ppm nitrite and ammonia as nitrogen source.

| | Time until outgrowth |
|---|---|
| Strain | [h] |
| DSM 33351 | 23.35 |
| DSM 33352 | 21.14 |
| DSM 33349 | 18.89 |
| DSM 33350 | 20.00 |

Spores of strains DSM 33351, DSM 33352, DSM 33349 and DSM 33350 were also tested regarding their ability to remove ammonia and nitrite in C minimal medium containing only glucose as carbon source and final concentration of 2.5 ppm of each nitrite and ammonia. 10 ml of the respective media in 100 ml shaking flasks (0.0352 g/l MnSO₄×1H₂O, 2.46 g/l MnSO₄×7H₂O, 0.2 KH₂PO₄, 0.6 g/l K₂HPO₄, 0.02 FeSO₄×7H₂O, 0.1396 g/l (NH₄)2SO₄, 0.00375 g/l NaNO₂, and 10 g/l Glucose (C:N ratio=20)) were inoculated with 10⁷ CFU/ml from the spore stocks. Cultures were incubated at 28° C. and 200 rpm. After 52 h, the concentrations of ammonia and nitrite were determined with the above described methods. The amount of ammonia and nitrite reduced by each strain was calculated referring to the initial concentration, which was determined in the non-inoculated control (Table 2.2). Ammonia and nitrite were removed efficiently from flasks inoculated with spores from strains DSM 33351, DSM 33352, DSM 33349 and DSM 33350. The strains were able to simultaneously remove minimum 99.6% of ammonia and 100% of nitrite within 52 h.

TABLE 2.2
Ammonia and nitrite reduction in C minimal medium containing
only glucose as C source and final concentration of 2.5 ppm
nitrite and ammonia as nitrogen source inoculated with spores.

| | *Removed ammonia [%]/ | *Removed nitrite [%]/ |
|---|---|---|
| | **Ammonia concentration | **Nitrite concentration |
| Strain* | [mg/l] | [mg/l] |
| DSM 33351 | 99.7 | 100.0 |
| DSM 33352 | 96.8 | 100.0 |
| DSM 33349 | 98.8 | 100.0 |
| DSM 33350 | 99.6 | 100.0 |
| Initial | 2.25 | 2.46 |
| concentration** | | |

These data show that spores of strains DSM 33351, DSM 33352, DSM 33349 and DSM 33350 are not only able to germinate and grow out in C minimal medium containing with 2.5 ppm nitrite and ammonia as sole nitrogen source, but they are also able to remove 2.5 ppm of each nitrite and ammonia within 52 h.

### Example 3: Ability of Strains to Inhibit Pathogens

The ability to inhibit different pathogens important in aqua culture was analyzed using well diffusion antagonism tests (Parente et al., 1995). Pathogens tested in these assays were Vibrio harveyi DSM 19623, Vibrio parahaemolyticus DSM 10027, Aeromonas hydrophila DSM 30187, Streptococcus agalactiae DSM 2134 and Flavobacterium columnare DSM 25092.

Vibrios are known to be associated with disease and high mortality in shrimp, but can also infect fish (Chatterjee and Haldar, 2012). Tilapia infections can be associated with Streptococcus agalactiae, a widely distributed bacterium, causing e.g. hemorrhage or erratic swimming (Mishra et al., 2018). Columnaris disease is caused by Flavobacterium columnare affecting fresh water fish. Salmon and trout farms, for instance, report high annual losses (Pulkkinen et al., 2010).

The pathogens were grown under appropriate conditions in liquid medium until an optical density of OD600 of at least 1.0 was reached. Pathogens were plated with a sterile spatula on the surface of Caso Yeast agar plates. Holes of 9 mm diameter were cut into the dried plates. Strains DSM 33351, DSM 33352, DSM 33349 and DSM 33350 were grown in LB-Kelly (LB Media supplemented with trace elements solution of DSMZ media 1032) for 16 h at 37° C. and 200 rpm in 100 ml shaking flasks. The cut wells were filled with non-inoculated media as control and the 100 μl of OD 5.0 adjusted cultures from strains DSM 33351, DSM 33352, DSM 33349 and DSM 33350. The plates were incubated under suitable conditions and the zone of clearance in mm was determined measuring from the edge of the cut well to the border of the cleared lawn. This zone was measured horizontally and vertically, and the average was taken. A scoring in low (+), medium (++), high (+++) and very high (++++) inhibition was applied according to Table 3.1.

TABLE 3.1
Scoring of pathogen inhibition based on diameter of hole
and clearing zone in well diffusion antagonism tests.

| Score | Diameter of hole + clearing zone [mm] |
|---|---|
| Low (+) | ≤9.5 mm |
| Medium (++), | >9.5 ≤ 15 |
| High (+++) | >15 ≤ 25 |
| Very high (++++) | >25 ≤ 30 |

Results for the inhibition of the pathogens by strains DSM 33351, DSM 33352, DSM 33349 and DSM 33350 can be found in Tables 3.2 to 3.6 respectively.

TABLE 3.2
Inhibitory capacity of B. subtilis DSM 33351 against different
pathogens in a well diffusion antagonism assays on Caso Yeast
medium, inhibition intensity was scored according to Table 4.1.

| Pathogen | Inhibition intensity |
|---|---|
| Vibrio harveyi DSM 19623 | + |
| Vibrio parahaemolyticus DSM 10027 | + |
| Aeromonas hydrophila DSM 30187 | ++ |
| Streptococcus agalactiae DSM 2134 | ++++ |

The data show that B. subtilis DSM 33351 can inhibit the growth of Vibrio harveyi DSM 19623, Vibrio parahaemolyticus DSM 10027, Aeromonas hydrophila DSM 30187 and Streptococcus agalactiae DSM 2134.

TABLE 3.3
Inhibitory capacity of B. subtilis DSM 33352 against different
pathogens in a well diffusion antagonism assays on Caso Yeast
medium, inhibition intensity was scored according to Table 5.1.

| Pathogen | Inhibition intensity |
|---|---|
| Aeromonas hydrophila DSM 30187 | ++ |
| Streptococcus agalactiae DSM 2134 | +++ |

The data show that B. subtilis DSM 33352 can inhibit the growth of Aeromonas hydrophila DSM 30187 and Streptococcus agalactiae DSM 2134.

TABLE 3.4
Inhibitory capacity of B. subtilis DSM 33349 against different
pathogens in a well diffusion antagonism assays on Caso Yeast
medium, inhibition intensity was scored according to Table 6.1.

| Pathogen | Inhibition intensity |
|---|---|
| Vibrio harveyi DSM 19623 | + |
| Aeromonas hydrophila DSM 30187 | ++ |
| Streptococcus agalactiae DSM 2134 | ++++ |
| Flavobacterium columnare DSM 25092 | + |

The data show that B. subtilis DSM 33349 can inhibit the growth of Vibrio harveyi DSM 19623, Aeromonas hydrophila DSM 30187, Streptococcus agalactiae DSM 2134 and Flavobacterium columnare DSM 25092.

TABLE 3.4
Inhibitory capacity of B. subtilis DSM 33350 against different
pathogens in a well diffusion antagonism assays on Caso Yeast
medium, inhibition intensity was scored according to Table 7.1.

| Pathogen | Inhibition intensity |
|---|---|
| Vibrio harveyi DSM 19623 | + |
| Vibrio parahaemolyticus DSM 10027 | + |
| Aeromonas hydrophila DSM 30187 | ++ |
| Streptococcus agalactiae DSM 2134 | ++++ |
| Flavobacterium columnare DSM 25092 | + |

The data show that B. subtilis DSM 33350 can inhibit the growth of Vibrio harveyi DSM 19623, Vibrio parahaemolyticus DSM 10027, Aeromonas hydrophila DSM 30187, Streptococcus agalactiae DSM 2134 and Flavobacterium columnare DSM 25092.

### LITERATURE

Parente, E., Brienza, C., Moles, M., & Ricciardi, A. (1995). A comparison of methods for the measurement of bacteriocin activity. Journal of microbiological methods, 22(1), 95-108.
Chatterjee, S. and Haldar, S. (2012). Vibrio Related Diseases in Aquaculture and Development of Rapid and Accurate Identification Methods. J. Marine Sci. Res. Dev. S1:002. doi:10.4172/2155-9910.S1-002.
Pulkkinen, K., Suomalainen, L.-R., Read, A. F., Ebert, D., Rintamäki, P. and Valtonen, E. T. (2010). Intensive fish farming and the evolution of pathogen virulence: the case of Columnaris disease in Finland. Proc. R. Soc. B. 277, 593-600.
Mishra, A., Nam, G. H., Gim, J. A., Lee, H. E., Jo, A. and Kim, H. S. (2018). Current Challenges of Streptococcus Infection and Effective Molecular, Cellular, and Environmental Control Methods in Aquaculture. Mol. Cells. 41(6):495-505

1-17. (canceled)

18. A Bacillus strain or a preparation thereof, wherein the strain or preparation is able to degrade at least one inorganic nitrogen compound and is further able to inhibit the growth of at least one pathogen.

19. The Bacillus strain or preparation thereof of claim 18, wherein the at least one pathogen is a pathogen of an aquatic animal.

20. The Bacillus strain or preparation thereof of claim 19, wherein the pathogen is selected from the group consisting of: Vibrio harveyi, Vibrio parahaemolyticus, Aeromonas hydrophila and Streptococcus agalactiae.

21. The Bacillus strain or preparation thereof of claim 18, wherein the at least one inorganic nitrogen compound is selected from the group consisting of: ammonia; nitrite; and nitrate.

22. The Bacillus strain or preparation thereof of claim 18, wherein the Bacillus strain or preparation thereof are selected from the group consisting of:
a) a Bacillus strain as deposited under one of the following numbers at the DSMZ: DSM 33349, DSM 33350, DSM 33351 and DSM 33352;
b) a mutant of a Bacillus strain in paragraph a) with a sequence identity to the strain of at least 95%;
c) a preparation of a strain according to paragraphs a) or b); and
d) a preparation comprising an effective mixture of compounds as contained in a strain as listed in paragraphs a) or b) or as contained in the preparation of paragraph c).

23. The Bacillus strain or preparation thereof of claim 22, wherein the Bacillus strain or preparation thereof are selected from the group consisting of:
a) a Bacillus strain as deposited under one of the following numbers at the DSMZ: DSM 33349, DSM 33350, DSM 33351 and DSM 33352;
b) a mutant of a Bacillus strain of paragraph a) with a sequence identity to the strain of at least 99%;
c) a preparation of a strain according to paragraphs a) or b);

24. The Bacillus strain or preparation thereof of claim 18, wherein the Bacillus strain has a 16S rDNA sequence with a sequence identity of at least 99% to a sequence according to SEQ ID NO: 1, SEQ ID NO: 6, SEQ ID NO: 11 or SEQ ID NO: 16.

25. The Bacillus strain or preparation thereof of claim 24, wherein the Bacillus strain or mutant thereof has a 16S rDNA sequence with a sequence identity of at least 99.8%, to a sequence according to SEQ ID NO: 1, SEQ ID NO: 6, SEQ ID NO: 11 or SEQ ID NO: 16.

26. The Bacillus strain of claim 18, wherein the strain is characterized by at least one of the following characteristics:
a) an ability to grow anaerobically;
b) an ability to grow in presence of 1 wt.-% of NaCl for at least one day;
c) an ability to germinate and/or to degrade inorganic nitrogen compounds in C minimal media;
d) an enzymatic activity selected from the group consisting of: cellulase activity; xylanase activity; protease activity; catalase activity; superoxide dismutase activity;
e) an ability to grow in presence of 2 mM bile.

27. The Bacillus strain of claim 26, wherein the strain is characterized by at least three, of characteristics a) to e).

28. The Bacillus strain or a preparation thereof claim 26, wherein the Bacillus strain or preparation thereof are selected from the group consisting of:
a) a Bacillus strain as deposited under one of the following numbers at the DSMZ: DSM 33349, DSM 33350, DSM 33351 and DSM 33352; or
b) a mutant of a Bacillus strain as listed in paragraph a) with a sequence identity to said strain of at least 99%.

29. The Bacillus strain or preparation thereof of claim 20, wherein the at least one inorganic nitrogen compound is selected from the group consisting of: ammonia; nitrite; and nitrate.

30. The Bacillus strain or preparation thereof of claim 29, wherein the Bacillus strain or preparation thereof are selected from the group consisting of:
a) a Bacillus strain as deposited under one of the following numbers at the DSMZ: DSM 33349, DSM 33350, DSM 33351 and DSM 33352;
b) a mutant of a Bacillus strain as listed in (a) with a sequence identity to the strain as listed in paragraph a) of at least 95%;
c) a preparation of a strain according to paragraphs a) or b);
d) a preparation comprising an effective mixture of compounds as contained in a strain as listed in paragraphs a) or b) or as contained in the preparation of paragraph.

31. The Bacillus strain or preparation thereof of claim 30, wherein the Bacillus strain or mutant thereof has a 16S rDNA sequence with a sequence identity of at least 99% to a sequence according to SEQ ID NO: 1, SEQ ID NO: 6, SEQ ID NO: 11 or SEQ ID NO: 16.

32. A method of decreasing the amount of inorganic nitrogen compounds in an aqueous system and/or controlling the amount of inorganic nitrogen compounds in an aqueous system, the method comprising supplying the aqueous system with at least one strain and/or at least one preparation of claim 18.

33. The method of claim 32, wherein the inorganic nitrogen compounds are selected from the group consisting of ammonium, nitrite and nitrate.

34. The method of claim 33, wherein the aqueous system is rearing water.

35. A method of feeding an animal, treating an animal for a disease or condition or preventing a disease or condition in an animal, comprising providing the animal with at least one strain and/or at least one preparation of claim 18.

36. The method of claim 35, wherein the animal is an aquatic animal.

37. The method of claim 36, wherein the animal is a crustacean or finfish.


SEQUENCE LISTING (25 sequences)

SEQ ID NO: 1 - DNA, 1457 bp, Bacillus subtilis
gttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 2 - DNA, 1197 bp, Bacillus subtilis
gtgaaaaataaatggctgtcttttttttcgggtaaggtccagcttgaattgacgggaagagggattgagcggctccttaatgaatgcacaagacaggggattccggtctttcatgtcaaaaaaaagaaagaagccgtatcgttatatatacagcttcaggatgtacatgcctttcggcgggtaagaagtaaatttaaatgtaaagcccgatttatcaatcggaagggatttcccttcctgttgctgaaatcaaagctgaatatagggtttacgatcggttttgcgatttttttcattcttttgtttttgctgtccaatatggtgtggaaaattgatgtgacaggcgctaagcctgaaacagaacatcaaatgaggcagcatcttaatgaaatcggcgtcaaaaagggccgtctgcagtttttaatgatgtcgcccgaaaaaatacagaaatcattaaccaatggaatagacaatatcacttgggtcggagttgatctgaaggggacgaccattcatatgaaagttgtggagaaaaatgagcccgaaaaagaaaaatatgttagcccgcgcaatattgtcgccaaaaagaaagcaaccattacgagaatgtttgtgcaaaaaggacagcccatggccgccatacacgatcatgttgaaaagggacagctgcttgtttcgggactgatcggcagcgaagaccatcagcaggaagtcgcctcaaaagcagaaatttatggagaaacctggtatagatcagaagtgacagtcccgcttgaaacattatttaacgtctatacgggcaaagtaaggacaaagcacaagctttcttttggttctttggcaatcccgatctgggggatgacgtttaaaaaagaggaattgaagcatccaaaaacagaacaagaaaagcattcgcttcattttctcggatttaagctccctgtatcctatgtcaaagagcaaacgagagaaagtgaagaggctttgcgaaaatatacaaaagaagaagcagttcaagaaggcattaaattgggtaaacaggatgtagaggataaaataggcgaaaacggcgaggtgaaaagtgaaaaagttttgcaccagactgttgagaatggtaaagtaaagttgattattctctaccaagttatagaagatatcgttcaaaccacacctattgtcagggagactgaagaatga

SEQ ID NO: 3 - DNA, 1923 bp, Bacillus subtilis
gtggctatggaacagcagcaaaacagttatgatgaaaatcagatacaggtactagaaggattggaagctgttcgtaaaagaccggggatgtatatcggttcgacaaacagcaaaggccttcaccacctggtatgggaaattgtcgacaatagtattgacgaagccctcgccggttattgtacggatatcaatatccaaatcgaaaaagacaacagtatcacggttgtagataatggccgcggtattccagtcggtattcatgaaaaaatgggccgtcctgcggtagaagtcattatgacggtacttcatgccggaggaaaatttgacggaagcggctataaagtatccggaggattacacggtgtaggtgcgtctgtcgtaaacgcactatcaacagagcttgatgtgacggttcaccgtgacggtaaaattcaccgccaaacttataaacgcggagttccggttacagaccttgaaatcattggcgaaacggatcatacaggaacgacgacacattttgtcccggaccctgaaattttctcagaaacaaccgagtatgattatgatctgcttgccaaccgcgtacgtgaattagcctttttaacaaagggcgtaaacatcacgattgaggataaacgtgaaggacaagagcgcaaaaatgaataccattacgaaggcggaattaaaagttatgtagagtatttaaaccgctctaaagaggttgtccatgaagagccgatttacattgaaggcgaaaaggacggcattacggttgaagtggctttgcaatacaatgacagctacacaagcaacatttactcgtttacaaacaacattaacacgtacgaaggcggtacccatgaagctggcttcaaaacgggcctgactcgtgttatcaacgattacgccagaaaaaaagggcttattaaagaaaatgatccaaacctaagcggagatgacgtaagggaagggctgacagcgattatttcaatcaaacaccctgatccgcagtttgagggccaaacgaaaacaaagctgggcaactcagaagcacggacgatcaccgatacgttattttctacggcgatggaaacatttatgctggaaaatccagatgcagccaaaaaaattgtcgataaaggcttaatggcggcaagagcaagaatggctgcgaaaaaagcccgtgaactaacacgtcgtaagagtgctttggaaatttcaaacctgcccggtaagttagcggactgctcttcaaaagatccgagcatctccgagttatatatcgtagagggtgactctgccggaggatctgctaaacaaggacgcgacagacatttccaagccattttgccgcttagaggtaaaatcctaaacgttgaaaaggccagactggataaaatcctttctaacaacgaagttcgctctatgatcacagcgctcggcacaggtattggggaagacttcaaccttgagaaagcccgttaccacaaagttgtcattatgacagatgccgatgttgacggcgcgcacatcagaacactgctgttaacgttcttttacagatatatgcgccaaattatcgagaatggctacgtgtacattgcgcagccgccgctctacaaggttcaacaggggaaacgcgttgaatatgcgtacaatgacaaggagcttgaagagctgttaaaaactcttcctcaaacccctaagcctggactgcagcgttacaaaggtcttggtgaaatgaatgccacccagctatgggagacaaccatggatcctagctccagaacacttcttcaggtaactcttgaagatgcaatggatgcggacgagacttttgaaatgcttatgggcgacaaggtagaaccgcgccgaaacttcatagaagcgaatgcgagatacgttaaaaatcttgacatctaa

SEQ ID NO: 4 - DNA, 3582 bp, Bacillus subtilis
ttgacaggtcaactagttcagtatggacgacaccgccagcgcagaagctatgctcgcattagcgaagtgttagaattaccaaatctcattgaaattcaaacctcttcttatcagtggtttcttgatgagggtcttagagagatgtttcaagacatatcaccaattgaggatttcactggtaacctctctcttgagttcattgattatagtttaggtgagcctaaatatcctgtagaggaatcaaaagaacgtgatgtgacttactcagctccgctaagagtgaaggttcgtttaattaacaaagaaactggagaggtaaaagaccaagatgtcttcatgggtgatttccctattatgacagatacaggtacttttatcattaacggtgcggaacgtgttattgtttcccagcttgttcggtctccaagtgtatatttcagtggtaaagtagacaaaaacggtaaaaaaggttttaccgcaactgtcattccaaaccgtggcgcatggttagaatacgaaactgatgcgaaagatgttgtttatgtccgcattgatcgcacacgtaagttgccggttacggttcttttgcgtgctctcggcttcggctccgatcaagagattcttgatctcataggagaaaacgaatacctgcgaaatacgcttgataaagataacacagaaaacagtgacaaagcgttgctggaaatttacgagcgtctccgtcctggagagccgcctacagtagaaaatgcgaaaagcttgcttgattctcgtttctttgatccgaaacgatacgatcttgccaatgtaggacgctataaaattaataaaaaacttcatattaagaatcgcctcttcaatcagagacttgctgaaacgcttgttgatcctgaaacaggagaaatccttgctgaaaaaggccagattcttgatagaagaacacttgataaagtactgccatacttagaaaacggaatcggttttagaaagctgtatccgaatggcggcgttgttgaagatgaagtgactcttcaatcaattaaaatctttgctccgactgatcaagaaggagaacaggttattaatgtaatcggcaatgcttacatcgaagaagagattaaaaacatcacgcctgctgatattatttcttcaatcagctacttcttcaacttgctgcacggagtaggcgatacagatgatatcgatcatcttggaaaccgccgtttacgctctgtaggcgagcttctccagaaccaattccgtatcggtttaagccgtatggaacgtgtggttcgtgagagaatgtcaattcaagatacgaatacaattacgcctcagcagctgatcaatattcgtcctgttattgcgtccattaaagagttctttggaagctcacagctttctcaattcatggatcagacgaacccgcttgctgaattaacgcacaagcgtcgtctgtcagcattaggaccgggcggattgacacgtgagcgtgccggaatggaagtgcgtgacgttcactactcccactatggccgtatgtgtccgattgaaacgcctgagggcccgaacatcggtttgatcaactcactatcatcttatgcaaaagtaaaccgttttggctttattgaaacgccatatcgccgcgttgaccctgaaacagggaaggtaacgggcagaatcgattacttaactgctgatgaagaggataactatgttgtcgctcaagcgaatgctcgtcttgatgacgaaggcgcctttattgatgacagcatcgtagctcgtttccgcggggagaacactgttgtttccagaaaccgtgtagactacatggatgtatcgcctaagcaggttgtatctgctgcgacagcatgtatcccgttcttagaaaacgatgactcgaaccgtgccctcatgggagcgaacatgcagcgccaggctgtgcctttgatgcagccggaagctccatttgttggaactggtatggaatacgtatcaggaaaagactctggtgccgctgttatttgtaaacaccctggtatcgttgaacgcgtagaagcgaaaaacgtttgggttcgccgttatgaagaagtagacggtcaaaaagtaaaaggaaacctggataaatacagcctgctgaaatttgtccgctctaaccaaggtacgtgctacaaccagcgtccgatcgtaagtgtcggcgatgaagtggtaaaaggagaaatccttgctgacggtccttctatggagcttggtgaacttgcacttggccgtaacgtaatggtcggcttcatgacatgggatggctacaactatgaggatgccatcatcatgagtgaacgcctagtgaaggatgatgtttatacatctatccacattgaagaatacgaatcagaagcacgtgatacgaaacttggacctgaagaaatcactcgcgatattccaaacgtcggtgaagatgcgcttcgcaatcttgatgaccgtggaatcatccgtattggggcagaagtaaaagacggagatcttcttgttggtaaagtaacgcctaaaggcgtaactgaactgactgcagaagaacgccttcttcacgccatttttggcgagaaagcccgcgaggttcgtgatacttctcttcgtgtgcctcacggcggcggcggaattatccatgacgttaaagtcttcaaccgtgaagacggagacgaacttcctccaggtgttaaccagttagtacgcgtatatatcgttcagaaacgtaagatttctgaaggggataaaatggccggtcgtcacggtaacaaaggtgttatctctaagattcttcctgaagaggatatgccttaccttcctgacggcacaccaattgatatcatgcttaacccgctgggcgtaccatcacgtatgaacatcgggcaggtattggaacttcacatgggtatggccgctcgttaccttggcattcacattgcatctcctgtatttgacggagcgcgagaagaggatgtctgggaaacacttgaagaagccggcatgtctcgtgacgccaaaacagtgctttacgacggacgtactggagagccgtttgataaccgtgtatctgtcggtatcatgtacatgatcaaactagctcacatggttgacgataaacttcatgcacgctctacaggtccttactcacttgttacgcagcagcctcttggcggtaaagcgcaatttggcggacagcgttttggtgagatggaggtttgggcacttgaagcttacggtgcggcttatactcttcaagaaattctgactgttaagtctgatgacgtggttggacgtgtgaaaacatacgaagccatcgttaaaggcgacaatgttcctgaaccaggtgttccggaatcattcaaagtattaatcaaagaacttcaaagcttaggtatggatgtcaaaatcctttctggtgatgaagaagaaatagaaatgagagatttagaagacgaagaagatgcgaaacaagctgacggcctggcattatcaggtgatgaagagccggaagaaacagcatctgcagacgttgaacgcgatgtagtaacaaaagaataa

SEQ ID NO: 5 - DNA, 1632 bp, Bacillus subtilis
atggcaaaagaaattaagtttagtgaagaagctcgccgcgcaatgcttcgcggtgtcgatgcacttgctgatgctgttaaagtaactttaggaccaaaaggacgcaacgtggttctagagaaaaaattcggttctccgttaatcacaaatgacggtgtaacaatcgctaaagaaatcgagctagaagacgcttttgaaaacatgggtgctaagcttgttgctgaagtagccagcaaaacaaacgacgttgccggtgacggtacaacaactgcaacagttcttgcgcaagcaatgatccgtgaaggccttaaaaacgtaacagcaggcgctaaccctgtaggcgtgcgtaaagggatggaacaagctgtagcggttgcgatcgaaaacttaaaagaaatttctaagccaatcgaaggcaaagagtctatcgctcaggttgctgcgatctctgctgctgacgaggaagtcggaagccttatcgctgaagcaatggagcgcgtaggaaacgacggcgttatcacaatcgaagagtctaaaggcttcacaactgagcttgaagttgttgaaggtatgcaattcgaccgcggatatgcgtctccttacatggtaactgactctgataagatggaagcggttcttgacaatccttacatcttaatcacagacaaaaaaatcacaaacattcaagaaatccttcctgtgcttgagcaggttgttcagcaaggcaaaccattgcttctgatcgctgaggatgttgaaggcgaagcacttgcaacacttgttgtgaacaaacttcgcggcacattcaacgcagttgctgttaaagctcctggcttcggtgaccgccgtaaagcaatgcttgaagacatcgctgtccttactggcggagaagtcatcacagaagatcttggccttgacctgaaatctactcaaatcgctcaattgggacgcgcttctaaagttgtcgttactaaagaaaacacaacaatcgttgaaggcgctggcgaaacagacaaaatttctgcccgcgtgactcaaatccgcgctcaagtggaagaaacaacttctgagttcgacagagaaaaattacaagagcgtcttgcgaaacttgctggcggcgtagctgtcatcaaagtcggcgctgcgactgaaactgagctgaaagagcgtaaacttcgcatcgaagacgccttgaactcaactcgcgcagctgttgaagaaggcatcgtatccggtggtggtacagcgcttgtaaacgtatataacaaagtcgctgcagttgaagctgaaggcgatgctcaaacaggtatcaacatcgtgcttcgcgcgcttgaagagccaatccgtcaaatcgcacacaacgctggccttgaaggatctgtcatcgttgagcgcctcaaaaacgaagaaatcggcgtaggcttcaacgctgcaactggcgaatgggtaaacatgatcgaaaaaggtatcgttgacccaacgaaagttacacgctcagctcttcaaaacgctgcgtctgtagctgcaatgttcttaactactgaagctgttgtcgctgacaagccagaagaaaacgctggcggcggaatgcctgacatgggcggcatgggcggtatgggcggcatgatgtaa

SEQ ID NO: 6 - DNA, 1547 bp, Bacillus subtilis
tcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 7 - DNA, 1197 bp, Bacillus subtilis
gtgaaaaataaatggctgtcttttttttcgggtaaggtccagcttgaattgacgggaagagggattgagcggctccttaatgaatgcacaagacaggggattccggtctttcatgtcaaaaaaaagaaagaagccgtatcgttatatatacagcttcaggatgtacatgcctttcggcgggtaagaagtaaatttaaatgtaaagcccgatttatcaatcggaagggatttcccttcctgttgctgaaatcaaagctgaatatagggtttacgatcggttttgcgatttttttcattcttttgtttttgctgtccaatatggtgtggaaaattgatgtgacaggcgctaagcctgaaacagaacatcaaatgaggcagcatcttaatgaaatcggcgtcaaaaagggccgtctgcagtttttaatgatgtcgcccgaaaaaatacagaaatcattaaccaatggaatagacaatatcacttgggtcggagttgatctgaaggggacgaccattcatatgaaagttgtggagaaaaatgagcccgaaaaagaaaaatatgttagcccgcgcaatattgtcgccaaaaagaaagcaaccattacgagaatgtttgtgcaaaaaggacagcccatggccgccatacacgatcatgttgaaaagggacagctgcttgtttcgggactgatcggcagcgaagaccatcagcaggaagtcgcctcaaaagcagaaatttacggagaaacctggtatagatcagaagtgacagtcccgcttgaaacattatttaacgtctatacgggcaaagtaaggacaaagcacaagctttcttttggttctttggcaatcccgatctgggggatgacgtttaaaaaagaggaattgaagcatccaaaaacagaacaagaaaagcattcgcttcattttctcggatttaagctccctgtatcctatgtcaaagagcaaacgagagaaagtgaagaggctttgcgaaaatatacgaaagaagaagcagttcaagaaggcattaaattgggtaaacaggatgtagaggataaaataggcgaaaacggcgaggtgaaaagtgaaaaagttttgcaccagactgttgagaatggtaaagtaaagttgattattctctaccaagttatagaagatatcgttcaaaccacacctattgtcagggagactgaagaatga

SEQ ID NO: 8 - DNA, 1923 bp, Bacillus subtilis
gtggctatggaacagcagcaaaacagttatgatgaaaatcagatacaggtactagaaggattggaagctgttcgtaaaagaccggggatgtatatcggttcgacaaacagcaaaggccttcaccacctggtatgggaaattgtcgacaatagtattgacgaagccctcgccggttattgtacggatatcaatatccaaatcgaaaaagacaacagtatcacggttgtagataatggccgcggtattccagtcggtattcatgaaaaaatgggccgtcctgcggtagaagtcattatgacggtacttcatgccggaggaaaatttgacggaagcggctataaagtatccggaggattacacggtgtaggtgcgtctgtcgtaaacgcactatcaacagagcttgatgtgacggttcaccgtgacggtaaaattcaccgccaaacttataaacgcggagttccggttacagaccttgaaatcattggcgaaacggatcatacaggaacgacgacacattttgtcccggaccctgaaattttctcagaaacaaccgagtatgattatgatctgcttgccaaccgcgtacgtgaattagcctttttaacaaagggcgtaaacatcacgattgaggataaacgtgaaggacaagagcgcaaaaatgaataccattacgaaggcggaattaaaagttatgtagagtatttaaaccgctctaaagaggttgtccatgaagagccgatttacattgaaggcgaaaaggacggcattacggttgaagtggctttgcaatacaatgacagctacacaagcaacatttactcgtttacaaacaacattaacacgtacgaaggcggtacccatgaagctggcttcaaaacgggcctgactcgtgttatcaacgattacgccagaaaaaaagggcttattaaagaaaatgatccaaacctaagcggagatgacgtaagggaagggctgacagcgattatttcaatcaaacaccctgatccgcagtttgagggccaaacgaaaacaaagctgggcaactcagaagcacggacgatcaccgatacgttattttctacggcgatggaaacatttatgctggaaaatccagatgcagccaaaaaaattgtcgataaaggcttaatggcggcaagagcaagaatggctgcgaaaaaagcccgtgaactaacacgtcgtaagagtgctttggaaatttcaaacctgcccggtaagttagcggactgctcttcaaaagatccgagcatctccgagttatatatcgtagagggtgactctgccggaggatctgctaaacaaggacgcgacagacatttccaagccattttgccgcttagaggtaaaatcctaaacgttgaaaaggccagactggataaaatcctttctaacaacgaagttcgctctatgatcacagcgctcggcacaggtattggggaagacttcaaccttgagaaagcccgttaccacaaagttgtcattatgacagatgccgatgttgacggcgcgcacatcagaacactgctgttaacgttcttttacagatatatgcgccaaattatcgaaaatggctacgtgtacattgcgcagccgccgctctacaaggttcaacaggggaaacgcgttgaatatgcgtacaatgacaaggagctggaagagctgttaaaaactcttcctcaaacgcctaagcctggactgcagcgttacaaaggtcttggtgaaatgaatgccacccagctatgggagacaaccatggatcctagttccagaacacttcttcaggtaactcttgaagatgcaatggatgcggacgagacttttgaaatgcttatgggcgacaaggtagaaccgcgccgaaacttcatagaagcgaatgcgagatacgttaaaaatcttgacatctaa

SEQ ID NO: 9 - DNA, 3582 bp, Bacillus subtilis
ttgacaggtcaactagttcagtatggacgacaccgccagcgcagaagctatgctcgcattagcgaagtgttagaattaccaaatctcattgaaattcaaacctcttcttatcagtggtttcttgatgagggtcttagagagatgtttcaagacatatcaccaattgaggatttcactggtaacctctctcttgagttcattgattatagtttaggtgagcctaaatatcctgtagaggaatcaaaagaacgtgatgtgacttactcagctccgctaagagtgaaggttcgtttaattaacaaagaaactggagaggtaaaagaccaagatgtcttcatgggtgatttccctattatgacagatacaggtacttttatcattaacggtgcggaacgtgttatcgtttcccagcttgttcggtctccaagtgtatatttcagtggtaaagtagacaaaaacggtaaaaaaggttttaccgcaactgtcattccaaaccgtggcgcatggttagaatacgaaactgatgcgaaagatgttgtttatgtccgcattgatcgcacacgtaagttgccggttacggttcttttgcgtgctctcggcttcggctccgatcaagagattcttgatctcataggagaaaacgaatacctgcgaaatacgcttgataaagataacacagaaaacagcgacaaagcgttgctggaaatttacgagcgtctccgtcctggagagccgcctacagtagaaaatgcgaaaagcttgcttgattctcgtttctttgatccgaaacgatacgatcttgccaatgtaggacgctataaaattaataaaaaacttcatattaagaatcgcctcttcaatcagagacttgctgaaacgcttgttgatcctgaaacaggagaaatccttgctgaaaaaggccagattcttgatagaagaacgcttgataaagtactgccatacttagaaaacggaatcggttttagaaagctgtatccgaatggcggcgttgttgaagatgaagtgactcttcaatcaattaaaatctttgctccgactgatcaagaaggagaacaggttatcaatgtaatcggcaatgcttacatcgaagaagagattaaaaacatcacgcctgctgatattatttcttcaatcagctacttcttcaacctgctgcacggagtaggcgacacagatgatatcgatcatcttggaaaccgccgtttacgttctgtaggcgagcttctccagaaccaattccgtatcggtttaagccgtatggagcgtgtggttcgtgagagaatgtcaattcaagatacgaatacaattacgcctcagcagctgatcaatattcgtcctgttattgcgtccattaaagagttctttggaagctcacagctttctcaattcatggatcagacgaacccgcttgctgaattaacgcacaagcgtcgtctgtcagcattaggaccgggcggattgacacgtgagcgtgccggaatggaagtgcgtgacgttcactactcccactatggccgtatgtgtccgattgaaacgcctgagggcccgaacatcggtttgatcaactcactatcatcttatgcaaaagtaaaccgttttggctttattgaaacgccatatcgccgcgttgaccctgaaacagggaaggtaacgggcagaatcgattacttaactgctgatgaagaggataactatgttgtcgctcaagcgaatgctcgtcttgatgacgaaggcgcctttattgatgacagcatcgtagctcgtttccgcggggagaacaccgttgtttccagaaatcgtgtagactacatggatgtatcgcctaagcaggttgtatctgctgcgacagcatgtatcccgttcttagaaaacgatgactccaaccgtgccctcatgggagcgaacatgcagcgtcaggctgtgcctttgatgcagccggaagcgccatttgttggaactggtatggaatacgtatcaggaaaagactctggtgccgctgttatttgtaaacaccctggtatcgttgaacgcgtagaagccaaaaacgtttgggttcgccgttatgaagaagtagacggtcaaaaagtaaaaggaaacctggataaatacagcctgctgaaatttgtccgctctaaccaaggtacgtgctacaaccagcgtccgatcgtaagtgtcggcgatgaagtggtaaaaggagaaatccttgctgacggtccttctatggagcttggtgaacttgcacttggccgtaacgtaatggtcggcttcatgacgtgggatggctacaactatgaggatgccatcatcatgagtgaacgcctagtgaaggatgatgtttatacatctatccacattgaagaatacgaatcagaagcacgtgatacgaaacttggacctgaagaaatcactcgcgatattccaaacgtcggtgaagatgcgcttcgcaatcttgatgaccgcggaatcatccgtattggggcagaagtaaaagacggagatcttcttgttggtaaagtaacgcctaaaggcgtaactgaactgactgccgaagaacgccttcttcacgccatctttggcgagaaagcccgcgaggttcgtgatacttctcttcgtgtgcctcatggcggcggcggaattatccatgacgttaaagtcttcaaccgtgaagacggagacgaacttcctccaggtgttaaccagttagtacgcgtatatatcgttcagaaacgtaagatttctgaaggggataaaatggccggtcgtcacggtaacaaaggtgttatctctaagattcttcctgaagaggatatgccttaccttcctgacggcacaccaattgatatcatgcttaacccgctgggcgtaccatcacgtatgaacatcgggcaggtattggaacttcacatgggtatggccgctcgttaccttggcattcacattgcatctcctgtatttgacggagcgcgagaagaggatgtctgggaaacacttgaagaagccggcatgtctcgtgacgccaaaacagtgctttacgacggacgtactggagagccgtttgataaccgtgtatctgtcggtatcatgtacatgatcaaactggctcacatggttgacgataaacttcatgcacgctctacaggtccttactcacttgttacgcagcagcctcttggcggtaaagcgcaatttggcggacagcgttttggtgagatggaggtttgggcacttgaagcttacggtgcggcttatactcttcaagaaattctgactgttaagtctgatgacgtggttggacgtgtgaaaacatacgaagccatcgttaaaggcgacaatgttcctgaaccaggtgttccggaatcattcaaagtattaatcaaagaacttcaaagcttaggtatggatgtcaaaatcctttctggtgatgaagaagaaatagaaatgagagatttagaagacgaagaagatgcgaaacaagctgacggcctggcattatcaggtgatgaagagccggaagaaacagcatctgcagacgttgaacgcgatgtagtaacaaaagaataa

SEQ ID NO: 10 - DNA, 1632 bp, Bacillus subtilis
atggcaaaagaaattaagtttagtgaagaagctcgccgcgcaatgcttcgcggtgtcgatgcacttgctgatgctgttaaagtaactttaggaccaaaaggacgcaacgtggttctagagaaaaaattcggttctccgttaatcacaaatgacggtgtaacaatcgctaaagaaatcgagctagaagacgcgtttgaaaacatgggtgctaagcttgttgctgaagtagccagcaaaacaaacgacgttgccggtgacggtacaacaactgcaacagttcttgcgcaagcaatgatccgtgaaggccttaaaaacgtaacagcaggcgctaaccctgtaggcgtgcgtaaagggatggaacaagctgtagcggttgcgatcgaaaacttaaaagaaatttctaagccaatcgaaggcaaagagtctatcgctcaggttgctgcgatctctgctgctgatgaggaagtcggaagccttatcgctgaagcaatggagcgcgtaggaaacgacggcgttatcacaatcgaagagtctaaaggcttcacaactgagcttgaagttgttgaaggtatgcaattcgaccgcggatatgcgtctccttacatggtaactgactctgataagatggaagcggttcttgacaatccttacatcttaatcacagacaaaaaaatcacaaacattcaagaaatccttcctgtgcttgagcaggttgttcagcaaggcaaaccattgcttctgatcgctgaggatgttgaaggcgaagcacttgcaacacttgttgtgaacaaacttcgcggcacattcaacgcagttgctgttaaagctcctggcttcggtgaccgccgtaaagcaatgcttgaagacatcgctgtccttactggcggagaagtcatcacagaagatcttggccttgacctgaaatctactcaaatcgctcaattgggacgcgcttctaaagttgtcgttactaaagaaaacacaacaatcgttgaaggcgctggcgaaacagacaaaatttctgcccgcgtgactcaaatccgcgctcaagtggaagaaacaacttctgaattcgacagagaaaaattacaagagcgtcttgctaaacttgctggcggcgtagctgtcatcaaagtcggcgctgcgactgaaactgagctgaaagagcgtaaacttcgcatcgaagacgccctgaactcaactcgcgcagctgttgaagaaggcatcgtatccggtggtggtacagcgcttgtaaacgtatataataaagtcgctgcagttgaagctgaaggcgatgctcaaacaggtatcaacatcgtgcttcgcgcgcttgaagagccaatccgtcaaatcgcacacaatgctggtcttgaaggatctgtcatcgttgaacgcctcaaaaacgaagaaatcggcgtaggcttcaacgctgcaactggcgaatgggtaaacatgatcgaaaaaggtatcgttgacccaacgaaagttacacgctcagctcttcaaaacgctgcgtctgtagctgcaatgttcttaacaactgaagccgttgtcgctgacaagccagaagaaaacgctggcggcggaatgcctgatatgggcggcatgggcggtatgggtggtatgatgtaa

SEQ ID NO: 11 - DNA, 1547 bp, Bacillus subtilis; identical to SEQ ID NO: 6

SEQ ID NO: 12 - DNA, 1197 bp, Bacillus subtilis; identical to SEQ ID NO: 2

SEQ ID NO: 13 - DNA, 1923 bp, Bacillus subtilis; identical to SEQ ID NO: 3

SEQ ID NO: 14 - DNA, 3582 bp, Bacillus subtilis
ttgacaggtcaactagttcagtatggacgacaccgccagcgcagaagctatgctcgcattagcgaagtgttagaattaccaaatctcattgaaattcaaacctcttcttatcagtggtttcttgatgagggtcttagagagatgtttcaagacatatcaccaattgaggatttcactggtaacctctctcttgagttcattgattatagtttaggtgagcctaaatatcctgtagaggaatcaaaagaacgtgatgtgacttactcagctccgctaagagtgaaggttcgtttaattaacaaagaaactggagaggtaaaagaccaagatgtcttcatgggtgatttccctattatgacagatacaggtacttttatcattaacggtgcggaacgcgttatcgtttcccagcttgttcggtctccaagtgtatatttcagtggtaaagtagacaaaaacggtaaaaaaggttttaccgcaactgtcattccaaaccgtggcgcatggttagaatacgaaactgatgcgaaagatgttgtttatgtccgcattgatcgcacacgtaagttgccggttacggttcttttgcgtgctctcggcttcggctccgatcaagagattcttgatctcataggagaaaacgaatacctgcgaaatacgcttgataaagataacacagaaaacagcgacaaagcgttgctggaaatttacgagcgtctccgtcctggagagccgcctacagtagaaaatgcgaaaagcttgcttgattctcgtttctttgatccgaaacgatacgatcttgccaatgtaggacgctataaaattaataaaaaacttcatattaagaatcgcctcttcaatcagagacttgctgaaacgcttgttgatcctgaaacaggagaaatccttgctgaaaaaggccagattcttgatagaagaacacttgataaagtactgccatacttagaaaacggaatcggttttagaaagctgtatccgaatggcggcgttgttgaagatgaagtgactcttcaatcaattaaaatctttgctccgactgatcaagaaggagaacaggttattaatgtaatcggcaatgcttacatcgaagaagagattaaaaacatcacgcctgctgatattatttcttcaatcagctacttcttcaacctgctgcacggagtaggcgacacagatgatatcgatcatcttggaaaccgccgtttacgttctgtaggcgagcttctccagaaccaattccgtatcggtttaagccgtatggagcgtgtggttcgtgagagaatgtcaattcaagatacgaatacaattacgcctcagcagctgatcaatattcgtcctgttattgcgtccattaaagagttctttggaagctcacagctttctcaattcatggatcagacgaacccgcttgctgaattaacgcacaagcgtcgtctgtcagcattaggaccgggcggattgacacgtgagcgtgccggaatggaagtgcgtgacgttcactactcccactatggccgtatgtgtccgattgaaacgcctgagggcccgaacatcggtttgatcaactcactatcatcttatgcaaaagtaaaccgttttggctttattgaaacgccatatcgccgcgttgatcctgaaacagggaaggtaacgggcagaatcgattacttaactgctgatgaagaggataactatgttgtcgctcaagcgaatgctcgtcttgatgacgaaggcgcctttattgatgacagcatcgtagctcgtttccgcggggagaacactgttgtttccagaaatcgtgtagactacatggatgtatcgcctaagcaggttgtatctgctgcgacagcatgtatcccgttcttagaaaacgatgactccaaccgtgccctcatgggagcgaacatgcagcgtcaggctgtgcctttaatgcagccggaagcgccatttgttggaactggtatggaatacgtatcaggaaaagactctggtgccgctgttatttgtaaacaccctggtatcgttgaacgcgtagaagcgaaaaacgtttgggttcgccgttatgaagaagtagacggtcaaaaagtaaaaggaaacctggataaatacagcctgctgaaatttgtccgctctaaccaaggtacgtgctacaaccagcgtccgatcgtaagtgtcggcgatgaagtggtaaaaggagaaatccttgctgacggtccttctatggagcttggtgaacttgcacttggccgtaacgtaatggtcggcttcatgacatgggatggctacaactatgaggatgccatcatcatgagtgaacgcctagtgaaggatgatgtttatacatctatccacattgaagaatacgaatcagaagcacgtgatacgaaacttggacctgaagaaatcactcgcgatattccaaatgtcggtgaagatgcgcttcgcaatcttgatgaccgtggaatcatccgtattggggcagaagtaaaagacggagatcttcttgttggtaaagtaacgcctaaaggcgtaactgaactgactgcagaagaacgccttcttcacgccatttttggcgagaaagcccgcgaggttcgtgatacttctcttcgtgtgcctcacggcggcggcggaattatccatgacgttaaagtcttcaaccgtgaagacggagacgaacttcctccaggtgttaaccagttagtacgcgtatatatcgttcagaaacgtaagatttctgaaggggataaaatggccggtcgtcacggtaacaaaggtgttatctctaagattcttcctgaagaggatatgccttaccttcctgacggcacaccaattgatatcatgcttaacccgctgggcgtaccatcacgtatgaacatcgggcaggtattggaacttcacatgggtatggccgctcgttaccttggcattcacattgcatctcctgtatttgacggagcgcgagaagaggatgtctgggaaacacttgaagaagccggcatgtctcgtgacgccaaaacagtgctttacgacggacgtactggagagccgtttgataaccgtgtatctgtcggtatcatgtacatgatcaaactggctcacatggttgacgataaacttcatgcacgctctacaggtccttactcacttgttacgcagcagcctcttggcggtaaagcgcaatttggcggacagcgttttggtgagatggaggtttgggcacttgaagcttacggtgcggcttatactcttcaagaaattctgactgttaagtctgatgacgtggttggacgtgtgaaaacatacgaagccatcgttaaaggcgacaatgttcctgaaccaggtgttccggaatcattcaaagtattaatcaaagaacttcaaagcttaggtatggatgtcaaaatcctttctggtgatgaagaagaaatagaaatgagagatttagaagacgaagaagatgcgaaacaagctgacggcctggcattatcaggtgatgaagagccggaagaaacagcatctgcagacgttgaacgcgatgtagtaacaaaagaataa

SEQ ID NO: 15 - DNA, 1632 bp, Bacillus subtilis
atggcaaaagaaattaagtttagtgaagaagctcgccgcgcaatgcttcgcggtgtcgatgcacttgctgatgctgttaaagtaactttaggaccaaaaggacgcaacgtggttctagagaaaaaattcggttctccgttaatcacaaatgacggtgtaacaatcgctaaagaaatcgagctagaagacgcgtttgaaaacatgggtgctaagcttgttgctgaagtagccagcaaaacaaacgacgttgcaggtgacggtacaacaactgcaacagttcttgcgcaagcaatgatccgtgaaggccttaaaaacgtaacagcaggcgctaaccctgtaggcgtgcgtaaagggatggaacaagctgtagcggttgcgatcgaaaacttaaaagaaatttctaagccaatcgaaggcaaagagtctatcgctcaggttgctgcgatctctgctgctgatgaggaagtcggaagccttatcgctgaagcaatggagcgcgtaggaaacgacggcgttatcacaatcgaagagtctaaaggcttcacaactgagcttgaagttgttgaaggtatgcaattcgaccgcggatatgcgtctccttacatggtaactgactctgataagatggaagcggttcttgacaatccttacatcttaatcacagacaaaaaaatcacaaacattcaagaaatccttcctgtgcttgagcaggttgttcagcaaggcaaaccattgcttctgatcgctgaggatgttgaaggcgaagcacttgcaacacttgttgtgaacaaacttcgcggcacattcaacgcagttgctgttaaagctcctggcttcggtgaccgccgtaaagcaatgcttgaagacatcgctgtccttactggcggagaagtcatcacagaagatcttggccttgacctgaaatctactcaaatcgctcaattgggacgcgcttctaaagttgtcgttactaaagaaaacacaacaatcgttgaaggcgctggcgaaacagacaaaatttctgcccgcgtgactcaaatccgcgctcaagtggaagaaacaacttctgaattcgacagagaaaaattacaagagcgtcttgctaaacttgctggcggcgtagctgtcatcaaagtcggcgctgcgactgaaactgaactgaaagagcgtaaacttcgcatcgaagacgccctgaactcaactcgcgcagctgttgaagaaggcatcgtatccggtggtggtacagcgcttgtaaacgtatataataaagtcgctgcagttgaagctgaaggcgatgctcaaacaggtatcaacatcgtgcttcgcgcgcttgaagagccaatccgtcaaatcgcacacaatgctggtcttgaaggatctgtcatcgttgaacgcctcaaaaacgaagaaatcggcgtaggcttcaacgctgcaactggcgaatgggtaaacatgatcgaaaaaggtatcgttgacccaacgaaagttacacgctcagctcttcaaaacgctgcgtctgtagctgcaatgttcttaacaactgaagccgttgtcgctgacaagccagaagaaaacgctggcggcggaatgcctgacatgggcggcatgggcggtatgggtggaatgatgtaa

SEQ ID NO: 16 - DNA, 1547 bp, Bacillus subtilis; identical to SEQ ID NO: 6

SEQ ID NO: 17 - DNA, 1197 bp, Bacillus subtilis; identical to SEQ ID NO: 2

SEQ ID NO: 18 - DNA, 1923 bp, Bacillus subtilis; identical to SEQ ID NO: 3

SEQ ID NO: 19 - DNA, 3582 bp, Bacillus subtilis; identical to SEQ ID NO: 4

SEQ ID NO: 20 - DNA, 1632 bp, Bacillus subtilis; identical to SEQ ID NO: 5

SEQ ID NO: 21 - DNA, 843 bp, Bacillus subtilis
atggctacaaatcaaacgcttaaaaacagcattcagaagaaacaaaacagtgccccaggacaacagcagggaacaacgatgaagggcctactttcttctccggcagtcatgaatcgattcgaagaagttctgggaaaaagagcgtctcaattcacagcgtcaattctgagcctgtacaacagtgaaaatacgcttcaaaaggcagaccctatgagtgtaatttcatcggctatggtggcggcaacgctcgatttgccagtggataagaacttaggttatgcctggatcgttccgtataaaggccgtgcgcaatttcagcttggttacaaaggatacattcagcttgctttgcggacgggccaatataaatctatcaattgcataccggttcatgaaggagaattgcaaaagtggaatccgttgaccgaggaaattgatattgattttgcaaagcgggaatcggagtcggttattggttatgctgcttatttcgaacttttaaatggattccgaaaaactgtctattggacaaaggcgcaagttgagaaacacaaaaagaaatttagtaagtcggacttcggttggggcaaggactgggatgcgatggcactcaaaaccgttctaaaatccatgctcagcaagtgggggattctctccgttgaaatgcaaaaggcagtcattgaggataacgaggaacgcgagcgaattgacattactgacgaaatgtctgagccagaaatcattgatgccgaaccatctgaggaaaaaccaagcgcgcaggatgctgatccttttgacggcaagcctgtagacatcagcgacgatgacctcccatttgattaa

SEQ ID NO: 22 - DNA, 825 bp, Bacillus subtilis
ttgttgaagtttaaaaacagaatagaagaaaaagaggttaagggggcagagaaggcgagctttggcgggcctaaccttctccacctgacacttcgcaaggttacgaagctatataagtatatcacagacaagaagtcttttccaatcctagttacctcattgtttgcattgtttaatcccaattcagttctagctgcagataagtatagtaattttgaagaattgaaagcaaatgagtcacctttgagctataacattttaactaccgatgttgataggtgtgtgcttattttggctcctcatgggggaggtattgaaggagggactagtgagcttgcaagagaactaagtaagtcttactctacctatctatttgaaggtttaagaataccaggagcttctgagttgcatattacaagtacgaattttgatgagcctaaagctttagatttattaagtaagcatgatttaacaatctcaattcatgggtatgcatcaagtaagaaacacactttagttggtgggacagacagagaaaaagcagcaaagattacatcattacttaccgatgcaggtttttctgcagaattgctgtctgaagattcccgtttagccggcacaaatgggcagaacattgctaataaaaatagtactggtatgagcattcaacttgaaataagcacagaacaacggagagaaatgttcaataattttacacttgatggacggaatggaacgcagagtcaagttttttatgattatattgctgttctaactaaattcattaatgaaaatgtgtattgtatggccggtgtagcaccatga

SEQ ID NO: 23 - DNA, 189 bp, Bacillus subtilis
atgaaaagaatcgaaaacccaaaacttaatgatgtaaaaattttaaaaaaggatgttagcagttttgcagtcgagttagaaaaaggtgataaagttcaaattatcggtattggtgaaagaggttatgatttgaagtatgttgacagtggagaagtcgtaaaagaatgtggatgggatttgtttgaataa

SEQ ID NO: 24 - DNA, 2802 bp, Bacillus subtilis
atggcaagtgttggagagttaacagctagaataaccttaggtggcatggatgctgtagaaagacagctcaagaactttagctctgctatggacaaagcttcacaaaaagcaggtgatatgggcaggactctcagggaagcaggtgagggcttcaatgagttaggggaaaacattgctcctttatcagcaggtttagtagctattggtggggcatctctagtagcctcagataacatcaatcaggcaatggctaactttcaaactaagttaggcgcatcaggatctgagttagcaaactatcagcaggtactaaatgatgtggctagcacaggagtaggctcttttgaagaggtctcagatggtatcataactgtttcacaaaacatgaaaggcttatctactggggagttaacaactctgtcagaacaggcaatgcagttaggtaaggtcatgggaactgatgtaggggatgtctcaaaagtagcaggacagttaatgactcagtttggtgtatcaggctcagaagctatggacttgattgctaagggttatcagaatggtatggattatgcaggggattatatggacactctgaatgagtactctgtctacttcaagacactaggattcagtggagaggacatgtttaacacccttatatcaggagcaaaagcaggggctttcaacttagataaagtgggagatgctgttaaggaatttggtatcaggtctaaggatggctcagagtcaactaaagaggcatttaaggcgcttggattagatgctgacaaaatgactcagactttcgcaaaaggtggcgaaggggctaagaaagggtatgctcaggtagttcaagccttatcaggtgttaaagatcaggctgacaggaatgctataggggtagccttgtttggtactcagtatgaggacatggaaaaggatgttattgcttccacaggtagcattgtagaccacatgggcaatgttaaaggtgcgagtgctgatgtagctaagaataacaagacctttgcacaggaaatgcagggagcatggaatcagattcagattgccattaagcctgtaggagatgtgctgaggggagctattgtagatgtaatgcctactatagtatctgcaatccaaagcatgtctaatgcctttatggggctatcacctacaatgcaaaaagttgtattaggtgtaggcgcttttgtagctatgatgcctgtaatgcttgcaggtattggaggtatcttgtctgtagtgggtactatggctacaggttggggagcattggcaagaggctttagcttagttgctacaggtggtacaaagcttgtatctgcattcaagctcataggcactgcctttagagctttgtctgcaatcttcatggctaacccattcatgctgattgtaacaggtattgtagttgtagctgtattgatctacaagtactgggatgaaatctcagcttacactatatctgcgtggaatgctattaaggcatggtttgctcctttttgggagtttttgaaatcattagtaagtgcttttgttactgccttcatgacttacattcaggctgtagttgccttttggacttctgtttggaatggcattaagactgtattcatggcaatatggtcaggactttcttccttttttttctcaatttggtcaggattgacagcagtagtgtctactgtattcaatgctatcaaagatgtcatccaaactgcatggaatgttataaagactgtgttccttattgccattgggattttagttacactgctagctcctttatggaatgcctttgctactgggtggaatgccttagtgggaattgtaaaggcagtaatgaataaggttaagtcatgggttaagacagcaatcaatttcatagtcactgcttggaaaaactatgtaaatgctgttaaaactgtgtggaatgcaatctatagcaatgtcattaagcctgtcatttcaaagattaagagcattgttaccactttgattgctaaggttaaaggggttatgaatgctgttaaaacagggtttaaagtggcttgggatgctatctacaatgctgtaataaaacctattctcaattacatcatcaatggcattaaaaatcttattagagccgcaaaatctgtaatgtcagcagtgaaatctgccttttctacagcttggaatgctattaagtctgtggtgacaaaggttatctcagtaatcaaaaatgtaatccaaggaattacttctaaagtgtcctcagtgacagggaaagtaaaatccttctttgtgaatgcatttaatgctgtgaaatctaaggtatcctcagctatctcaagtgtgaaagataagattagcagtatctggaatacagcaaagtctatagcaggaaaaatcaagggtgcttttggtaacatcttcaagagcattaaaatccctcacttttcaatgggtgggtggactgttaaggacttaccaaaactgcctaagatgtcaattagttggcacgcaaaagggggtatcttggacagctctactctcattggtgcaggtgagaaaggtgctgaggcaattgtacctctgtcctctcaaagaaggatgaagccatttgctcaagcagtgtctaagtttatgcctgattctgcaaaaggtagctctaatacttccattcatgtaggacagttggtagtaagagaggaagcagatattcagaggattgcacaagagctgaatagattgcaagacaagaaggagagggctaaaggtaggctctcctttgcttaa

SEQ ID NO: 25 - DNA, 216 bp, Bacillus subtilis
atgaaagactttacaactatgggctgtctagcaatcatttttattggcttgcttgtagcagtgggtataggtgtcaactttttgctgatgccttacctactaatgattgctctaggagcttttggagtccacttctcttacctagtatgtattgcattatggatagtagtaacagtattgctaagaaagctattgccaagcagaaaagctgaatag